(19) United States
(12) Patent Application Publication
(10) Pub. No.: US 2025/0292612 A1
(43) Pub. Date: Sep. 18, 2025

(54) DETECTION DEVICE

(71) Applicant: Japan Display Inc., Tokyo (JP)

(72) Inventor: Gen KOIDE, Tokyo (JP)

(21) Appl. No.: 19/220,373

(22) Filed: May 28, 2025

Related U.S. Application Data
(63) Continuation of application No. PCT/JP2023/042845, filed on Nov. 30, 2023.

(30) Foreign Application Priority Data
Nov. 30, 2022 (JP) 2022-191869

(51) Int. Cl. G06V 40/13; H04N 23/56; H04N 25/76; H04N 25/772; H10F 55/00

(52) U.S. Cl. CPC G06V 40/1318; H04N 23/56; H04N 25/772; H04N 25/7795; H10F 55/18

(57) ABSTRACT

According to an aspect, a detection device includes: an optical sensor; a first light source configured to emit first light to the optical sensor; a second light source configured to emit second light different from the first light to the optical sensor; and a detection circuit configured to acquire a first detection value when the first light is emitted to the optical sensor and a second detection value when the second light is emitted to the optical sensor. A light emission period of the second light source is relatively shorter than a light emission period of the first light source.

### CROSS-REFERENCE TO RELATED APPLICATION

This application claims the benefit of priority from Japanese Patent Application No. 2022-191869 filed on Nov. 30, 2022 and International Patent Application No. PCT/JP2023/042845 filed on Nov. 30, 2023, the entire contents of which are incorporated herein by reference.

### BACKGROUND

### 1. Technical Field

What is disclosed herein relates to a detection device.

### 2. Description of the Related Art

Detection devices are known that emit light into a body through the skin thereof and acquire an oxygen saturation level in blood (hereinafter, called “blood oxygen saturation level (SpO₂)”) based on transcutaneous data acquired by detecting light transmitted through or reflected by arteries. The blood oxygen saturation level (SpO₂) refers to a ratio of an amount of oxygen actually bound to hemoglobin to the total amount of oxygen under the assumption that the oxygen is bound to all the hemoglobin in the blood. When acquiring the blood oxygen saturation level (SpO₂), for example, a pulse wave acquired using red light and a pulse wave acquired using infrared light are used (refer to Japanese Patent Application Laid-open Publication No. 2019-180861, for example).

In calculating the blood oxygen saturation level (SpO₂), the pulse wave acquired using the red light and the pulse wave acquired using the infrared light are used. Therefore, a difference in detection timing between a first detection value detected by using the red light and a second detection value detected by using the infrared light is preferably smaller.

For the foregoing reasons, there is a need for a detection device capable of reducing the difference in detection timing between the first detection value and the second detection value that are used to calculate the blood oxygen saturation level (SpO₂).

### SUMMARY

According to an aspect, a detection device includes: an optical sensor; a first light source configured to emit first light to the optical sensor; a second light source configured to emit second light different from the first light to the optical sensor; and a detection circuit configured to acquire a first detection value when the first light is emitted to the optical sensor and a second detection value when the second light is emitted to the optical sensor. A light emission period of the second light source is relatively shorter than a light emission period of the first light source.

### BRIEF DESCRIPTION OF THE DRAWINGS

FIG. 1 is a plan view illustrating a detection device according to a first embodiment of the present disclosure;

FIG. 2 is a block diagram illustrating a configuration example of the detection device according to the first embodiment;

FIG. 3 is a circuit diagram illustrating the detection device;

FIG. 4 is a circuit diagram illustrating a plurality of partial detection areas;

FIG. 5A is a sectional view illustrating a schematic sectional configuration of a sensor area;

FIG. 5B is a sectional view illustrating a schematic sectional configuration of the sensor area of a detection device according to a first modification of the present disclosure;

FIG. 6 is a timing waveform diagram illustrating an operation example of the detection device;

FIG. 7 is a timing waveform diagram illustrating an operation example in a reset period in FIG. 6;

FIG. 8 is a timing waveform diagram illustrating an operation example in a readout period in FIG. 6;

FIG. 9 is a timing waveform diagram illustrating an operation example in a drive period of one gate line included in the readout period in FIG. 6;

FIG. 10 is an explanatory diagram for explaining a relation between driving of the sensor area and lighting operations of light sources in the detection device;

FIG. 11 is a plan view schematically illustrating a relation between the sensor area, first light sources, and second light sources in the detection device according to the embodiment;

FIG. 12 is a side view of the detection device illustrated in FIG. 11 as viewed in a first direction Dx;

FIG. 13 is an explanatory diagram for explaining an operation example of the detection device according to a first technical example according to the first embodiment;

FIG. 14 is a timing waveform diagram illustrating the operation example of the detection device according to the first technical example to be compared to the first embodiment;

FIG. 15 is a conceptual diagram illustrating a relation between an amount of current flowing in a light source and an emission intensity thereof;

FIG. 16 is an explanatory diagram for explaining an operation example of the detection device according to the first embodiment;

FIG. 17 is an explanatory diagram for explaining an operation example of the detection device according to a second technical example according to the first embodiment;

FIG. 18 is an explanatory diagram for explaining an operation example of the detection device according to a modification of the first embodiment;

FIG. 19 is a block diagram illustrating an exemplary circuit configuration of the detection device according to a second embodiment of the present disclosure;

FIG. 20 is an explanatory diagram for explaining an operation example of the detection device according to a comparative example to the second embodiment;

FIG. 21 is an explanatory diagram for explaining an operation example of the detection device according to the second embodiment;

FIG. 22 is a block diagram illustrating an exemplary circuit configuration of the detection device according to a third embodiment of the present disclosure;

FIG. 23 is an explanatory diagram for explaining an operation example of the detection device according to a comparative example to the third embodiment; and

FIG. 24 is an explanatory diagram for explaining an operation example of the detection device according to the third embodiment.

### DETAILED DESCRIPTION

The following describes modes (embodiments) for carrying out the present disclosure in detail with reference to the drawings. The present disclosure is not limited to the description of the embodiments given below. Components described below include those easily conceivable by those skilled in the art or those substantially identical thereto. In addition, the components described below can be combined as appropriate. What is disclosed herein is merely an example, and the present disclosure naturally encompasses appropriate modifications easily conceivable by those skilled in the art while maintaining the gist of the disclosure. To further clarify the description, the drawings may schematically illustrate, for example, widths, thicknesses, and shapes of various parts as compared with actual aspects thereof. However, they are merely examples, and interpretation of the present disclosure is not limited thereto. The same component as that described with reference to an already mentioned drawing is denoted by the same reference numeral through the present specification and the drawings, and detailed description thereof may not be repeated where appropriate.

In this disclosure, when an element is described as being “on” another element, the element can be directly on the other element, or there can be one or more elements between the element and the other element.

### First Embodiment

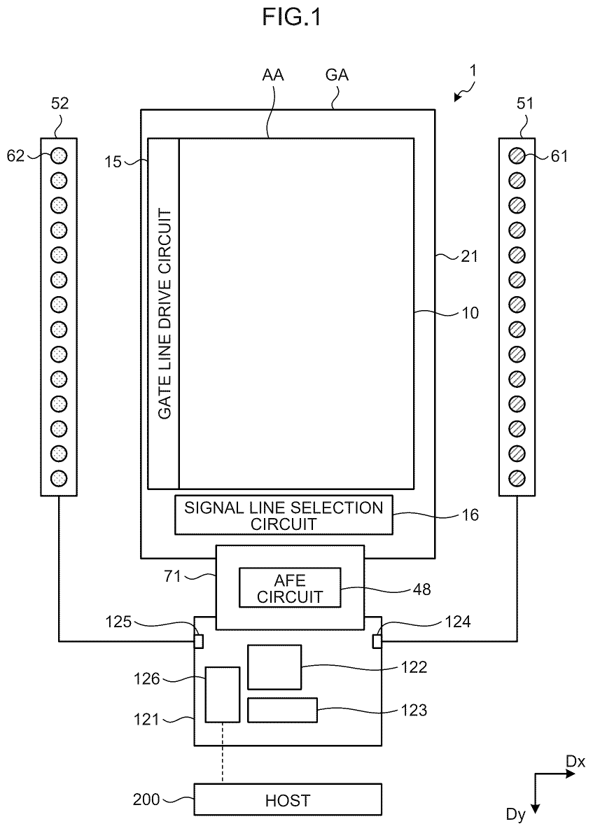

FIG. 1 is a plan view illustrating a detection device according to a first embodiment of the present disclosure. As illustrated in FIG. 1, a detection device 1 includes a sensor base member 21, a sensor area 10, a gate line drive circuit 15, a signal line selection circuit 16, an analog front-end (AFE) circuit 48, a control circuit 122, a power supply circuit 123, first light sources 61, and second light sources 62. FIG. 1 illustrates an example in which a first light source base member 51 is provided with the first light sources 61 and a second light source base member 52 is provided with the second light sources 62. However, the arrangement of the first and the second light sources 61 and 62 illustrated in FIG. 1 is merely exemplary, and can be modified as appropriate. For example, the first and the second light sources 61 and 62 may be arranged on each of the first and the second base members 51 and 52. In this case, a group including the first light sources 61 and a group including the second light sources 62 may be arranged in a second direction Dy, or the first and the second light sources 61 and 62 may be alternately arranged in the second direction Dy. The first and the second light sources 61 and 62 may be provided on one light source base member, or three or more light source base members. A specific example of the arrangement of the first and the second light sources 61 and 62 will be described later.

The detection device 1 is electrically coupled to a host 200. The host 200 is, for example, a higher-level control device for an apparatus (not illustrated) to which the detection device 1 is applied. The host 200 performs a predetermined biometric information acquisition process based on data output from the detection device 1.

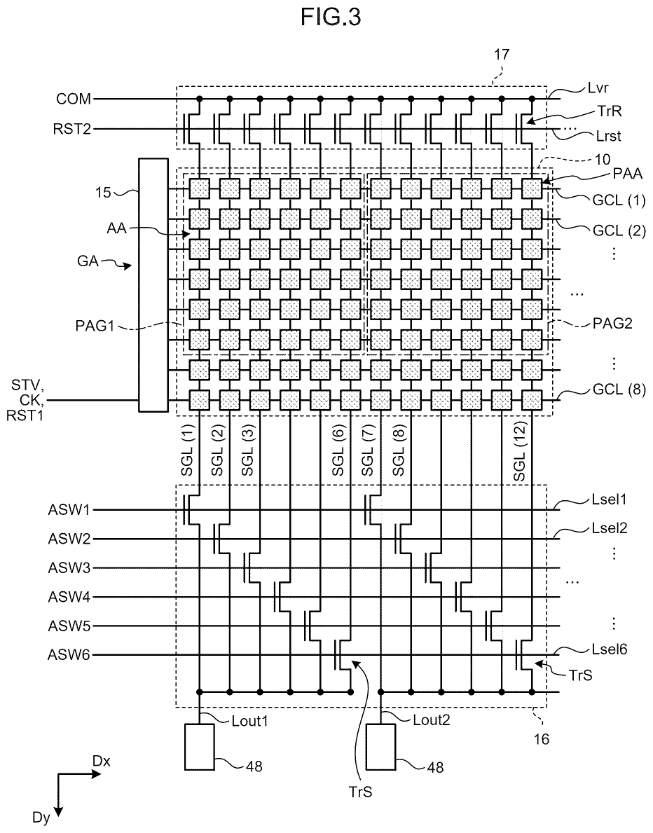

The sensor base member 21 is electrically coupled to a control substrate 121 via a flexible printed circuit board 71. The flexible printed circuit board 71 is provided with the AFE circuit 48. The control substrate 121 is provided with the control circuit 122, the power supply circuit 123, and an output circuit 126. As illustrated in FIG. 3 described later, the AFE circuit 48 is circuitry including a plurality of AFE circuits each of which is provided for a plurality signal lines. In the following descriptions, each of the plurality of AFE circuits included in the AFE circuit 48 as entire circuitry is given the same reference sign “48” and is referred to as the “AFE circuit 48” in some cases.

The control circuit 122 is, for example, a control integrated circuit (IC) that outputs logic control signals. The control circuit 122 may be, for example, a programmable logic device (PLD) such as a field-programmable gate array (FPGA).

The control circuit 122 supplies control signals to the sensor area 10, the gate line drive circuit 15, and the signal line selection circuit 16 to control detection operations in the sensor area 10. The control circuit 122 also supplies control signals to the first and the second light sources 61 and 62 to control lighting or non-lighting of the first and the second light sources 61 and 62.

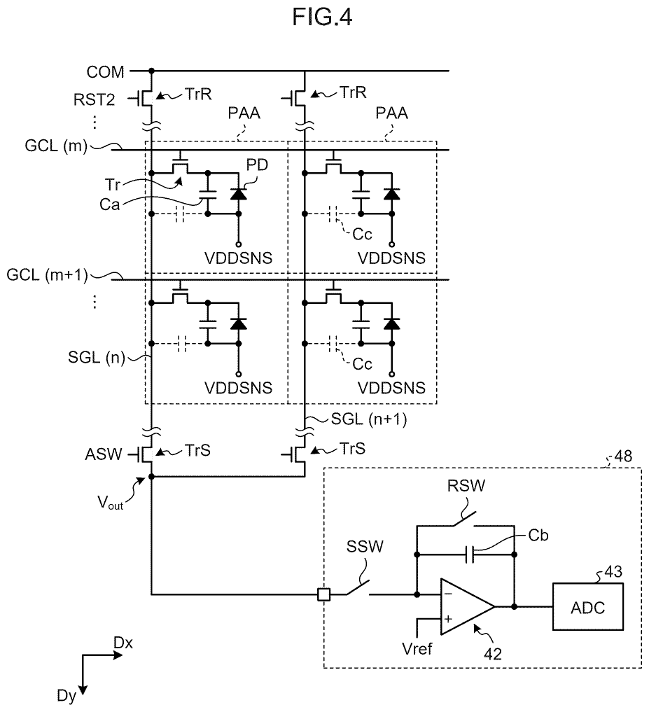

The power supply circuit 123 supplies voltage signals such as a sensor power supply potential VDDSNS (refer to FIG. 4) to the sensor area 10, the gate line drive circuit 15, and the signal line selection circuit 16. The power supply circuit 123 supplies a power supply voltage to the first and the second light sources 61 and 62.

The output circuit 126 is, for example, a Universal Serial Bus (USB) controller IC, and controls communication between the control circuit 122 and the host 200.

The sensor base member 21 has a detection area AA and a peripheral area GA. The detection area AA is an area where a plurality of optical sensors PD (refer to FIG. 4) included in the sensor area 10 are provided in a matrix having a row-column configuration. The peripheral area GA is an area between the outer perimeter of the detection area AA and the ends of the sensor base member 21 and is an area not provided with the optical sensors PD.

The gate line drive circuit 15 and the signal line selection circuit 16 are provided in the peripheral area GA. Specifically, the gate line drive circuit 15 is provided in an area extending along the second direction Dy in the peripheral area GA. The signal line selection circuit 16 is provided in an area extending along a first direction Dx in the peripheral area GA and is provided between the sensor area 10 and the AFE circuit 48.

The first direction Dx is one direction in a plane parallel to the sensor base member 21. The second direction Dy is one direction in the plane parallel to the sensor base member 21, and is a direction orthogonal to the first direction Dx. The second direction Dy may non-orthogonally intersect the first direction Dx. A third direction Dz is a direction orthogonal to the first direction Dx and the second direction Dy, and is a direction normal to the sensor base member 21.

The first light sources 61 are provided on the first light source base member 51, and arranged along the second direction Dy. The second light sources 62 are provided on the second light source base member 52, and arranged along the second direction Dy. The first light source base member 51 and the second light source base member 52 are electrically coupled, through respective terminals 124 and 125 provided on the control substrate 121, to the control circuit 122 and the power supply circuit 123.

For example, inorganic light-emitting diodes (LEDs) or organic electroluminescent (EL) diodes (organic light-emitting diodes (OLEDs)) are used as the first and the second light sources 61 and 62. The first and the second light sources 61 and 62 emit first and second light, respectively, having different wavelengths.

The first light emitted from the first light sources 61 is reflected, for example, on a surface of an object to be detected, such as a finger or a wrist of a subject, and is incident on the sensor area 10. As a result, the sensor area 10 can detect a fingerprint by detecting a shape of asperities on the surface of a finger Fg or the like. The second light emitted from the second light sources 62 is, for example, reflected in the finger Fg or the like, or transmitted through the finger Fg or the like, and is incident on the sensor area 10. As a result, the sensor area 10 can detect information on a living body in the finger, the wrist, or the like of the subject. Examples of the information on the living body include, but are not limited to, pulse waves, pulsation, and a vascular image of the subject. That is, the detection device 1 may be configured as a fingerprint detection device that detects the fingerprint or a vein detection device that detects a vascular pattern of, for example, veins.

The first light may have a wavelength of 420 nm to 600 nm, for example, approximately 500 nm, and the second light may have a wavelength of 780 nm to 950 nm, for example, approximately 850 nm. In this case, the first light is blue or green visible light (blue light or green light), and the second light is infrared light. The sensor area 10 can detect the fingerprint based on the first light emitted from the first light sources 61. The second light emitted from the second light sources 62 is reflected in, or transmitted through or absorbed by the object to be detected, and is incident on the sensor area 10. As a result, the sensor area 10 can detect biometric data such as the pulse waves and the vascular image (vascular pattern) as the information on the living body in the finger, the wrist, or the like of the subject.

Alternatively, the first light may have a wavelength of 600 nm to 700 nm, for example, approximately 660 nm, and the second light may have a wavelength of 780 nm to 950 nm, for example, approximately 850 nm. In this case, the sensor area 10 can detect a blood oxygen level in addition to the pulse waves, the pulsation, and the vascular image as the information on the living body based on the first light emitted from the first light sources 61 and the second light emitted from the second light sources 62. In this way, the detection device 1 includes the first and the second light sources 61 and 62, and performs the detection based on the first light and the detection based on the second light, and thereby can detect the various types of information on the living body.

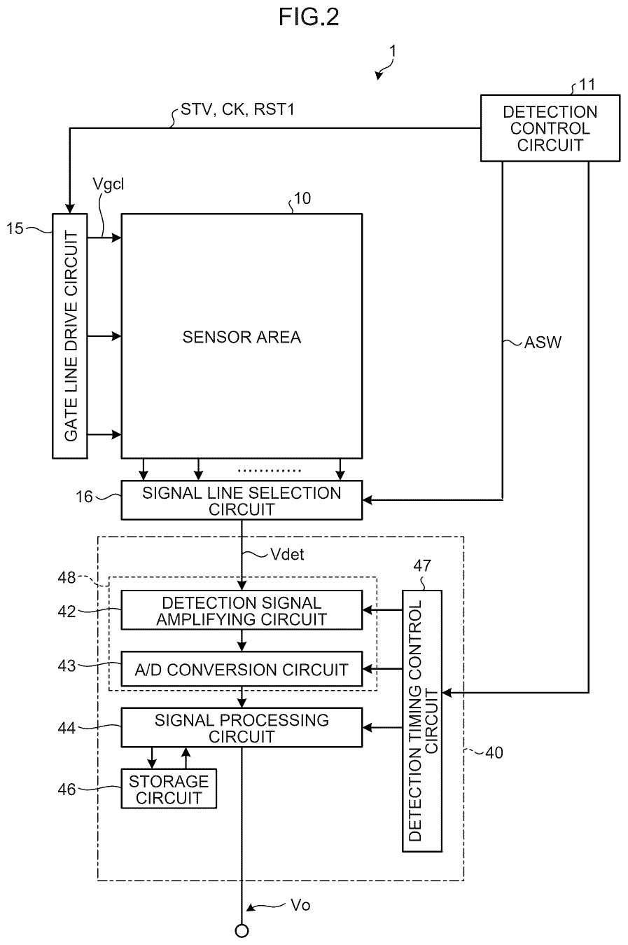

FIG. 2 is a block diagram illustrating a configuration example of the detection device according to the first embodiment. As illustrated in FIG. 2, the detection device 1 further includes a detection control circuit 11 and a detection circuit 40.

The sensor area 10 includes the optical sensors PD. Each of the optical sensors PD included in the sensor area 10 is an organic photodiode (OPD) and outputs an electrical signal corresponding to light received by the optical sensor PD as a detection signal Vdet to the signal line selection circuit 16. The sensor area 10 performs detection in response to a gate drive signal Vgcl supplied from the gate line drive circuit 15.

The detection control circuit 11 is a circuit that supplies respective control signals to the gate line drive circuit 15, the signal line selection circuit 16, and the detection circuit 40 to control operations of these circuits. The detection control circuit 11 supplies various control signals such as a start signal STV, a clock signal CK, and a reset signal RST1 to the gate line drive circuit 15. The detection control circuit 11 also supplies various control signals such as a selection signal ASW to the signal line selection circuit 16. The detection control circuit 11 also supplies various control signals to the first and the second light sources 61 and 62 to control the lighting and the non-lighting of each group of the first and the second light sources 61 and 62.

The gate line drive circuit 15 is a circuit that drives a plurality of gate lines GCL (refer to FIG. 3) based on the various control signals. The gate line drive circuit 15 sequentially or simultaneously selects the gate lines GCL and supplies the gate drive signals Vgcl to the selected gate lines GCL. By this operation, the gate line drive circuit 15 selects the optical sensors PD coupled to the gate lines GCL.

The signal line selection circuit 16 is a switch circuit that sequentially or simultaneously selects a plurality of signal lines SGL (refer to FIG. 3). The signal line selection circuit 16 is a multiplexer, for example. The signal line selection circuit 16 electrically couples the selected signal lines SGL to the AFE circuit 48 based on the selection signals ASW supplied from the detection control circuit 11. Through this operation, the signal line selection circuit 16 outputs the detection signals Vdet of the optical sensors PD to the detection circuit (detection processing circuit) 40.

The detection circuit 40 includes the AFE circuit 48, a signal processing circuit 44, a storage circuit 46, and a detection timing control circuit 47. The detection timing control circuit 47 controls the AFE circuit 48 and the signal processing circuit 44 to operate in synchronization with each other based on the control signal supplied from the detection control circuit 11.

The AFE circuit 48 generates a detection value of each of the optical sensors PD based on the detection signal of the optical sensor PD output from the sensor area 10. The AFE circuit 48 is an analog front-end (AFE) circuit, for example.

The AFE circuit 48 is a signal processing circuit having functions of at least a detection signal amplifying circuit 42 and an analog-to-digital (A/D) conversion circuit 43. The detection signal amplifying circuit 42 amplifies the detection signals Vdet. The A/D conversion circuit 43 converts analog signals output from the detection signal amplifying circuit 42 into digital signals.

In the present disclosure, the control circuit 122 includes the signal processing circuit 44 and the storage circuit 46.

The signal processing circuit 44 acquires the biometric data for generating the information on the living body based on the detection values of the optical sensors PD output from the AFE circuit 48. In the present disclosure, the information on the living body includes the pulse waves acquired using the infrared light and red light.

The storage circuit 46 temporarily stores therein the signals processed by the signal processing circuit 44. In the present disclosure, the storage circuit 46 stores therein a biometric data acquisition area and various types of setting information that are set in a biometric data acquisition area setting process flow (to be described later) when the signal processing circuit 44 acquires the biometric data. The storage circuit 46 may have a configuration including a random-access memory (RAM), a read-only memory (ROM), an electrically erasable programmable read-only memory (EEPROM), and the like. The storage circuit 46 may be a register circuit or the like.

The following describes a circuit configuration example of the detection device 1. FIG. 3 is a circuit diagram illustrating the detection device. As illustrated in FIG. 3, the sensor area 10 has a plurality of partial detection areas PAA arranged in a matrix having a row-column configuration. Each of the partial detection areas PAA is provided with the optical sensor PD.

The gate lines GCL extend in the first direction Dx and are each coupled to the partial detection areas PAA arranged in the first direction Dx. A plurality of gate lines GCL(1), GCL(2), . . . , GCL(8) are arranged in the second direction Dy and are each coupled to the gate line drive circuit 15. In the following description, the gate lines GCL(1), GCL(2), . . . , GCL(8) will each be simply referred to as the gate line GCL when they need not be distinguished from one another. To facilitate understanding of the description, FIG. 3 illustrates eight gate lines GCL. However, this is merely an example, and M gate lines GCL may be arranged (where M is a natural number, such as 256).

The signal lines SGL extend in the second direction Dy and are each coupled to the optical sensors PD of the partial detection areas PAA arranged in the second direction Dy. A plurality of signal lines SGL(1), SGL(2), . . . , SGL(12) are arranged in the first direction Dx and are each coupled to the signal line selection circuit 16 and a reset circuit 17. In the following description, the signal lines SGL(1), SGL(2), . . . , SGL(12) will each be simply referred to as the signal line SGL when they need not be distinguished from one another.

To facilitate understanding of the description, 12 signal lines SGL are illustrated. However, this is merely an example, and N signal lines SGL may be arranged (where N is a natural number, such as 252). In FIG. 3, the sensor area 10 is provided between the signal line selection circuit 16 and the reset circuit 17. The signal line selection circuit 16 and the reset circuit 17 are not limited to being provided in this way, and may be coupled to ends of the signal lines SGL on the same side.

The gate line drive circuit 15 receives the various control signals such as the start signal STV, the clock signal CK, and the reset signal RST1 from the control circuit 122 (refer to FIG. 1). The gate line drive circuit 15 sequentially selects the gate lines GCL(1), GCL(2), . . . , GCL(8) in a time-division manner based on the various control signals. The gate line drive circuit 15 supplies the gate drive signal Vgcl to the selected one of the gate lines GCL. This operation supplies the gate drive signal Vgcl to a plurality of first switching elements Tr coupled to the gate line GCL, and thus selects the partial detection areas PAA arranged in the first direction Dx as detection targets.

The gate line drive circuit 15 may perform different driving for each of detection modes including the detection of the fingerprint and the detection of a plurality of different items of information on the living body (such as the pulse waves, the pulsation, the vascular image, and the blood oxygen level, which are hereinafter called also simply “biometric information”). For example, the gate line drive circuit 15 may collectively drive more than one of the gate lines GCL.

Specifically, the gate line drive circuit 15 simultaneously selects a predetermined number of the gate lines GCL from among the gate lines GCL(1), GCL(2), . . . , GCL(8) based on the control signals. For example, the gate line drive circuit 15 simultaneously selects six gate lines GCL(1) to GCL(6) and supplies thereto the gate drive signals Vgcl. The gate line drive circuit 15 supplies the gate drive signals Vgcl via the selected six gate lines GCL to the first switching elements Tr. This operation selects block units PAG1 and PAG2 each including more than one of the partial detection areas PAA arranged in the first direction Dx and the second direction Dy as the detection targets. The gate line drive circuit 15 collectively drives the predetermined number of the gate lines GCL, and sequentially supplies the gate drive signals Vgcl to each unit of the predetermined number of the gate lines GCL.

The signal line selection circuit 16 includes a plurality of selection signal lines Lsel, a plurality of output signal lines Lout, and third switching elements TrS. The third switching elements TrS are provided correspondingly to the signal lines SGL. Six signal lines SGL(1), SGL(2), . . . , SGL(6) are coupled to a common output signal line Lout1. Six signal lines SGL(7), SGL(8), . . . , SGL(12) are coupled to a common output signal line Lout2. The output signal lines Lout1 and Lout2 are each coupled to the AFE circuit 48.

The signal lines SGL(1), SGL(2), . . . , SGL(6) are grouped into a first signal line block, and the signal lines SGL(7), SGL(8), . . . , SGL(12) are grouped into a second signal line block. The selection signal lines Lsel are coupled to the gates of the respective third switching elements TrS included in one of the signal line blocks. One of the selection signal lines Lsel is coupled to the gates of the third switching elements TrS in the signal line blocks.

Specifically, selection signal lines Lsel1, Lsel2, . . . , Lsel6 are coupled to the third switching elements TrS corresponding to the signal lines SGL(1), SGL(2), . . . , SGL(6), respectively. The selection signal line Lsel1 is coupled to one of the third switching elements TrS corresponding to the signal line SGL(1) and one of the third switching elements TrS corresponding to the signal line SGL(7). The selection signal line Lsel2 is coupled to one of the third switching elements TrS corresponding to the signal line SGL(2) and one of the third switching elements TrS corresponding to the signal line SGL(8).

The control circuit 122 (refer to FIG. 1) sequentially supplies the selection signals ASW to the selection signal lines Lsel. This operation causes the signal line selection circuit 16 to operate the third switching elements TrS to sequentially select the signal lines SGL in one of the signal line blocks in a time-division manner. The signal line selection circuit 16 selects one of the signal lines SGL in each of the signal line blocks. Such a configuration can reduce the number of integrated circuits (ICs) including the AFE circuit 48 or the number of terminals of the ICs in the detection device 1.

The signal line selection circuit 16 may collectively couple more than one of the signal lines SGL to the AFE circuit 48. Specifically, the control circuit 122 (refer to FIG. 1) simultaneously supplies the selection signals ASW to the selection signal lines Lsel. The signal line selection circuit 16 operates the third switching elements TrS to select the signal lines SGL (for example, six signal lines SGL) in one of the signal line blocks, and couples the signal lines SGL to the AFE circuit 48. As a result, signals detected in each of the block units PAG1 and PAG2 are output to the AFE circuit 48. In this case, the signals from the partial detection areas PAA (optical sensors PD) included in each of the block units PAG1 and PAG2 are integrated and output to the AFE circuit 48.

The detection is performed for each of the block units PAG1 and PAG2 by the operations of the gate line drive circuit 15 and the signal line selection circuit 16. As a result, the strength of the detection signal Vdet obtained by a one-time detection operation increases, so that the sensor sensitivity can be improved.

In the present disclosure, the detection device 1 can change the number of the partial detection areas PAA (optical sensors PD) included in each of the block units PAG1 and PAG2. Thus, the value of resolution per inch (pixels per inch (ppi), hereinafter, referred to as “definition”) can be set depending on the information to be acquired.

For example, the number of the partial detection areas PAA (optical sensors PD) included in each of the block units PAG1 and PAG2 is relatively reduced. While this setting results in a longer detection time and a lower frame rate (for example, 20 frames per second (fps) or lower), the detection can be performed at a higher definition (for example, at 300 ppi or higher). Hereafter, the term “first mode” denotes a mode of performing the detection at a lower frame rate and a higher definition. By selecting the first mode of performing the detection at a lower frame rate and a higher definition, for example, the fingerprint on the surface of the finger can be acquired at a higher definition.

Alternatively, for example, the number of the partial detection areas PAA (optical sensors PD) included in each of the block units PAG1 and PAG2 is relatively increased. While this setting results in a lower definition (for example, 50 ppi or lower), the detection can be performed at a higher frame rate (for example, at 100 fps or higher) that allows the detection to be repeatedly performed in a shorter time in one frame. Hereafter, the term “second mode” denotes a mode of performing the detection at a higher frame rate and a lower definition. By selecting the second mode of performing the detection at a higher frame rate and a lower definition, for example, a change in pulse wave over time can be more accurately detected. In the second mode, calculation of a pulse wave velocity, calculation of blood pressure, and the like are enabled by using the pulse waves acquired at a higher frame rate (for example, 1000 fps or higher).

For example, when acquiring the vascular image (vein pattern), the number of the partial detection areas PAA (optical sensors PD) included in each of the block units PAG1 and PAG2 is set to an intermediate value between those of the first mode and the second mode. This setting allows the detection to be performed at a medium frame rate that is higher than that of the first mode and lower than that of the second mode (for example, higher than 20 fps and lower than 100 fps) and at a medium definition that is lower than that of the first mode and higher than that of the second mode (for example, higher than 50 ppi and lower than 300 ppi). Hereafter, the term “third mode” denotes a mode of performing the detection at the medium frame rate and the medium definition. The third mode of performing the detection at the medium frame rate and the medium definition is suitable, for example, for acquiring the vascular pattern of veins and the like.

As illustrated in FIG. 3, the reset circuit 17 includes a reference signal line Lvr, a reset signal line Lrst, and fourth switching elements TrR. The fourth switching elements TrR are provided correspondingly to the signal lines SGL. The reference signal line Lvr is coupled to either the sources or the drains of the fourth switching elements TrR. The reset signal line Lrst is coupled to the gates of the fourth switching elements TrR.

The control circuit 122 supplies a reset signal RST2 to the reset signal line Lrst. This operation turns on the fourth switching elements TrR to electrically couple the signal lines SGL to the reference signal line Lvr. The power supply circuit 123 supplies a reference signal COM to the reference signal line Lvr. This operation supplies the reference signal COM to a capacitive element Ca (refer to FIG. 4) included in each of the partial detection areas PAA.

FIG. 4 is a circuit diagram illustrating the partial detection areas of the detection device according to the embodiment. FIG. 4 also illustrates a circuit configuration of the AFE circuit 48. As illustrated in FIG. 4, each of the partial detection areas PAA includes the optical sensor PD, the capacitive element Ca, and a corresponding one of the first switching elements Tr1. The capacitive element Ca is capacitance (sensor capacitance) generated in the optical sensor PD, and is equivalently coupled in parallel to the optical sensor PD. In addition, signal line capacitance Cc is parasitic capacitance generated on the signal line SGL, and is equivalently provided between the signal line SGL, and the anode of the optical sensor PD and one end side of the capacitive element Ca.

FIG. 4 illustrates two gate lines GCL(m) and GCL(m+1) arranged in the second direction Dy among the gate lines GCL. FIG. 4 also illustrates two signal lines SGL(n) and SGL(n+1) arranged in the first direction Dx among the signal lines SGL. The partial detection area PAA is an area surrounded by the gate lines GCL and the signal lines SGL.

Each of the first switching elements Tr is provided correspondingly to the optical sensor PD. The first switching element Tr is made of a thin-film transistor, and in this example, made of an n-channel metal oxide semiconductor (MOS) thin-film transistor (TFT).

The gates of the first switching elements Tr belonging to the partial detection areas PAA arranged in the first direction Dx are coupled to the gate line GCL. The sources of the first switching elements Tr belonging to the partial detection areas PAA arranged in the second direction Dy are coupled to the signal line SGL. The drain of the first switching element Tr is coupled to the cathode of the optical sensor PD and the capacitive element Ca.

The anode of the optical sensor PD is supplied with the sensor power supply signal (sensor power supply potential) VDDSNS from the power supply circuit 123. The signal line SGL and the capacitive element Ca are supplied with the reference signal COM serving as an initial potential of the signal line SGL and the capacitive element Ca from the power supply circuit 123.

When the partial detection area PAA is irradiated with light, a current corresponding to an amount of light flows through the optical sensor PD. As a result, an electric charge corresponding to the amount of light is stored in the capacitive element Ca. When the first switching element Tr is turned on, a current corresponding to the electric charge stored in the capacitive element Ca flows through the signal line SGL. The signal line SGL is coupled to the AFE circuit 48 through a corresponding one of the third switching elements TrS of the signal line selection circuit 16. Thus, the detection device 1 can detect a signal corresponding to the amount of light irradiating the optical sensor PD in each of the partial detection areas PAA or signals corresponding to the amounts of light irradiating the optical sensors PD in each of the block units PAG1 and PAG2.

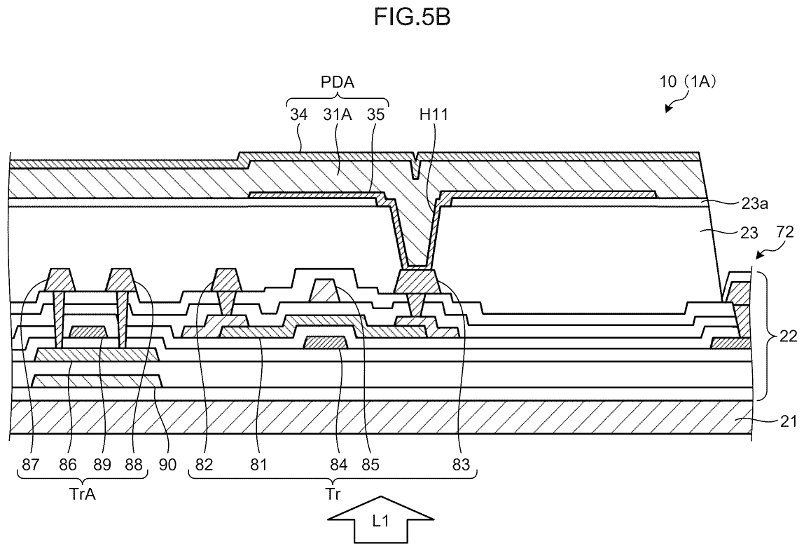

During a readout period Pdet (refer to FIG. 6), a switch SSW of the AFE circuit 48 is turned on to couple the AFE circuit 48 to the signal line SGL. The detection signal amplifying circuit 42 of the AFE circuit 48 converts a current supplied from the signal line SGL into a voltage corresponding to the value of the current and amplifies the result. A reference potential (Vref) having a fixed potential is supplied to a non-inverting input terminal (+) of the detection signal amplifying circuit 42, and the signal line SGL is coupled to an inverting input terminal (−) of the detection signal amplifying circuit 42. In the present embodiment, the same signal as the reference signal COM is supplied as the reference potential (Vref) voltage. The detection signal amplifying circuit 42 includes a capacitive element Cb and a reset switch RSW. During a reset period Prst (refer to FIG. 6), the reset switch RSW is turned on, and the electric charge of the capacitive element Cb is reset.

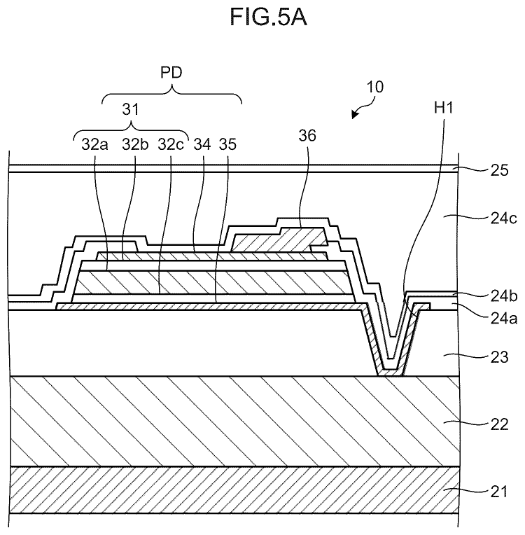

The following describes a configuration of the optical sensor PD. FIG. 5A is a sectional view illustrating a schematic sectional configuration of the sensor area. As illustrated in FIG. 5A, the sensor area 10 includes the sensor base member 21, a TFT layer 22, an insulating layer 23, the optical sensor PD, and insulating layers 24a, 24b, 24c, and 25. The sensor base member 21 is an insulating base member, and is made using, for example, glass or a resin material. The sensor base member 21 is not limited to having a flat plate shape, and may have a curved surface. In this case, the sensor base member 21 may be made of a film-like resin. The sensor base member 21 has a first surface and a second surface opposite the first surface. The TFT layer 22, the insulating layer 23, the optical sensor PD, and the insulating layers 24 and 25 are stacked in this order on the first surface.

The TFT layer 22 is provided with circuits such as the gate line drive circuit 15 and the signal line selection circuit 16 described above. The TFT layer 22 is also provided with thin-film transistors (TFTs), such as the first switching elements Tr, and various types of wiring, such as the gate lines GCL and the signal lines SGL. The sensor base member 21 and the TFT layer 22 serve as a drive circuit board that drives the sensor for each predetermined detection area, and are also called a backplane or an array substrate.

The insulating layer 23 is an organic insulating layer, and is provided on the TFT layer 22. The insulating layer 23 is a planarizing layer that planarizes asperities formed by the first switching elements Tr and various conductive layers formed in the TFT layer 22.

The optical sensor PD is provided on the insulating layer 23. The optical sensor PD includes a lower electrode 35, a semiconductor layer 31, and an upper electrode 34, which are stacked in this order.

The lower electrode 35 is provided on the insulating layer 23, and is electrically coupled to the first switching element Tr in the TFT layer 22 through a contact hole Hi. The lower electrode 35 is the cathode of the optical sensor PD and is an electrode for reading out the detection signal Vdet. A metal material such as molybdenum (Mo) or aluminum (Al) is used as the lower electrode 35. Alternatively, the lower electrode 35 may be a multilayered film formed by stacking these metal materials. The lower electrode 35 may be formed, for example, of a light-transmitting conductive material such as indium tin oxide (ITO).

The semiconductor layer 31 is formed of amorphous silicon (a-Si). The semiconductor layer 31 includes an i-type semiconductor layer 32a, a p-type semiconductor layer 32b, and an n-type semiconductor layer 32c. The i-type semiconductor layer 32a, the p-type semiconductor layer 32b, and the n-type semiconductor layer 32c are a specific example of a photoelectric conversion element. In FIG. 5A, the n-type semiconductor layer 32c, the i-type semiconductor layer 32a, and the p-type semiconductor layer 32b are stacked in this order in a direction orthogonal to a surface of the sensor base member 21. However, the semiconductor layer 31 may have a reversed configuration, that is, the p-type semiconductor layer 32b, the i-type semiconductor layer 32a, and the n-type semiconductor layer 32c may be stacked in this order. The semiconductor layer 31 may be a photoelectric conversion element formed of organic semiconductors.

The a-Si of the n-type semiconductor layer 32c is doped with impurities to form an n+ region. The a-Si of the p-type semiconductor layer 32b is doped with impurities to form a p+ region. The i-type semiconductor layer 32a is, for example, a non-doped intrinsic semiconductor, and has lower conductivity than that of the p-type semiconductor layer 32b and the n-type semiconductor layer 32c.

The upper electrode 34 is the anode of the optical sensor PD, and is an electrode for supplying the power supply signal VDDSNS to the photoelectric conversion layers. The upper electrode 34 is a light-transmitting conductive layer of, for example, ITO, and is provided in common to all the optical sensors PD.

The insulating layers 24a and 24b are provided on the insulating layer 23. The insulating layer 24a covers the periphery of the upper electrode 34, and is provided with an opening at a location overlapping the upper electrode 34. Coupling wiring 36 is coupled to the upper electrode 34 at a portion of the upper electrode 34 not provided with the insulating layer 24a. The insulating layer 24b is provided on the insulating layer 24a so as to cover the upper electrode 34 and the coupling wiring 36. The insulating layer 24c serving as a planarizing layer is provided on the insulating layer 24b. The insulating layer 25 is provided on the insulating layer 24c. However, the insulating layer 25 need not be provided.

FIG. 5B is a sectional view illustrating a schematic sectional configuration of the sensor area of a detection device according to a first modification of the present disclosure. As illustrated in FIG. 5B, in a detection device 1A of the first modification, an optical sensor PDA is provided on an insulating layer 23a. The insulating layer 23a is an inorganic insulating layer provided so as to cover the insulating layer 23, and is formed of silicon nitride (SiN), for example. The optical sensor PDA includes a photoelectric conversion layer 31A, the lower electrode 35 (cathode electrode), and the upper electrode 34 (anode electrode). The lower electrode 35, the photoelectric conversion layer 31A, and the upper electrode 34 are stacked in this order in a direction orthogonal to a first surface Si of the sensor base member 21.

The photoelectric conversion layer 31A changes in characteristics (for example, voltage-current characteristics and resistance value) depending on light emitted thereto. An organic material is used as a material of the photoelectric conversion layer 31A. Specifically, as the photoelectric conversion layer 31A, low-molecular-weight organic materials can be used, such as fullerene (C₆₀), phenyl-C₆₁-butyric acid methyl ester (PCBM), copper phthalocyanine (CuPc), fluorinated copper phthalocyanine (F₁₆CuPc), 5,6,11,12-tetraphenyltetracene (rubrene), and perylene diimide (PDI) (derivative of perylene).

The photoelectric conversion layer 31A can be formed by a vapor deposition process (dry process) using any of the low-molecular-weight organic materials listed above. In this case, the photoelectric conversion layer 31A may be, for example, a multilayered film of CuPc and F₁₆CuPc, or a multilayered film of rubrene and C₆₀. The photoelectric conversion layer 31A can also be formed by a coating process (wet process). In this case, the photoelectric conversion layer 31A is made using a material obtained by combining any of the above-listed low-molecular-weight organic materials with a high-molecular-weight organic material. As the high-molecular-weight organic material, for example, poly(3-hexylthiophene) (P3HT) and F8-alt-benzothiadiazole (F8BT) can be used. The photoelectric conversion layer 31A can be a film in the state of a mixture of P3HT and PCBM, or a film in the state of a mixture of F8BT and PDI.

The lower electrode 35 faces the upper electrode 34 with the photoelectric conversion layer 31A interposed therebetween. A light-transmitting conductive material such as ITO is used as the upper electrode 34. A metal material such as silver (Ag) or aluminum (Al) is used as the lower electrode 35. Alternatively, the lower electrode 35 may be an alloy material containing at least one or more of these metal materials.

The lower electrode 35 can be formed as a light-transmitting transflective electrode by controlling the film thickness of the lower electrode 35. For example, the lower electrode 35 is formed of a thin Ag film having a thickness of 10 nm so as to have a light-transmitting property of approximately 60%. In this case, the optical sensor PDA can detect light emitted from both sides of the sensor base member 21, for example, both light Li emitted from the first surface Si side and light emitted from a second surface S2 side.

Although not illustrated in FIG. 5B, the insulating layer 24 may be provided so as to cover the upper electrode 34. The insulating layer is a passivation film, and is provided to protect the optical sensor PDA.

As illustrated in FIG. 5B, the TFT layer 22 is provided with the first switching element Tr electrically coupled to the optical sensor PDA. The first switching element Tr includes a semiconductor layer 81, a source electrode 82, a drain electrode 83, and gate electrodes 84 and 85. The lower electrode 35 of the optical sensor PDA is electrically coupled to the drain electrode 83 of the first switching element Tr through a contact hole H11 provided in the insulating layers 23 and 23a.

The first switching element Tr has what is called a dual-gate structure provided with the gate electrodes 84 and 85 on both the upper and lower sides of the semiconductor layer 81. However, the first switching element Tr is not limited to this structure and may have a top-gate structure or a bottom-gate structure.

FIG. 5B schematically illustrates a second switching element TrA and a terminal 72 that are provided in the peripheral area GA. The second switching element TrA is, for example, a switching element provided in the gate line drive circuit 15 (refer to FIG. 1). The second switching element TrA includes a semiconductor layer 86, a source electrode 87, a drain electrode 88, and a gate electrode 89. The second switching element TrA has what is called a top-gate structure provided with the gate electrode 89 on the upper side of the semiconductor layer 86. A light-blocking layer 90 is provided between the semiconductor layer 86 and the sensor base member 21 on the lower side of the semiconductor layer 86. However, the second switching element TrA is not limited to this structure, and may have a bottom-gate structure or a dual-gate structure.

The semiconductor layer 81 of the first switching element Tr is provided in a layer different from that of the semiconductor layer 86 of the second switching element TrA. The semiconductor layer 81 of the first switching element Tr is formed of an oxide semiconductor, for example. The semiconductor layer 86 of the second switching element TrA is formed of polysilicon, for example.

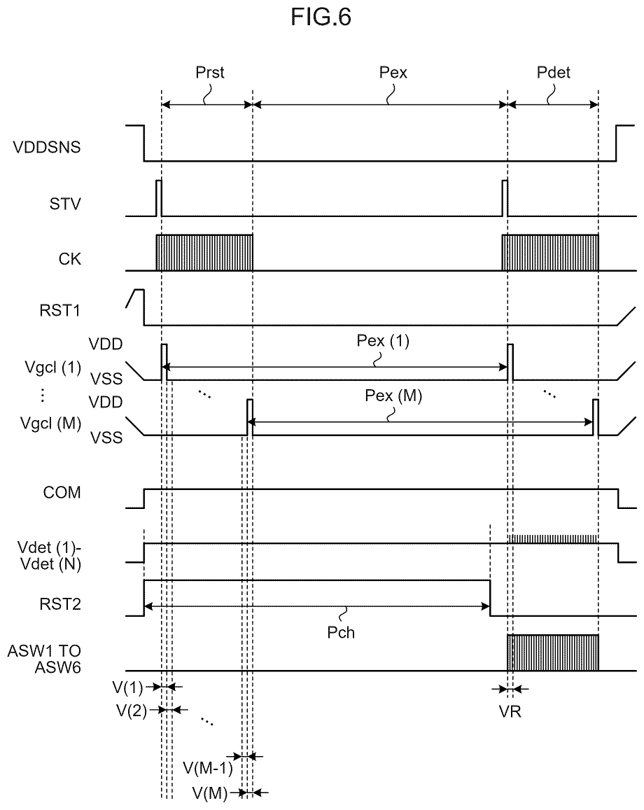

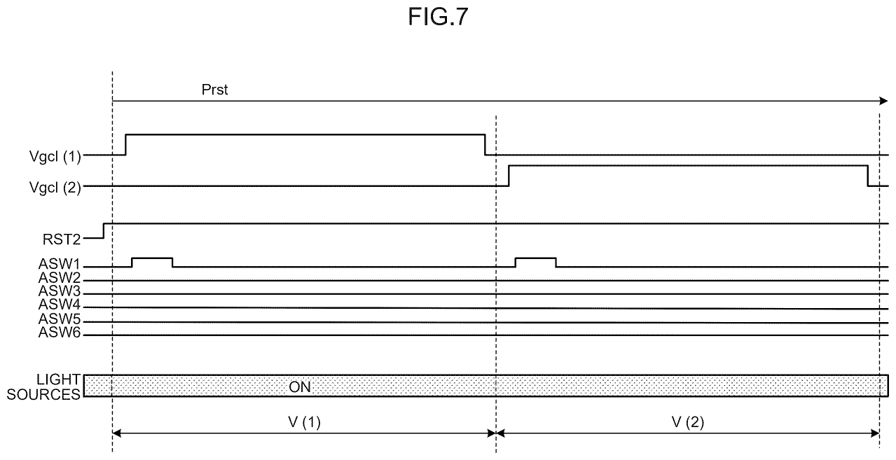

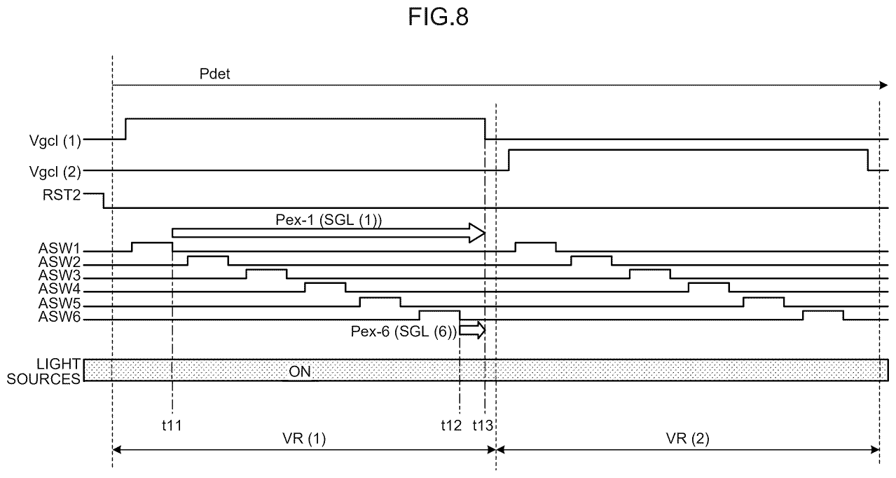

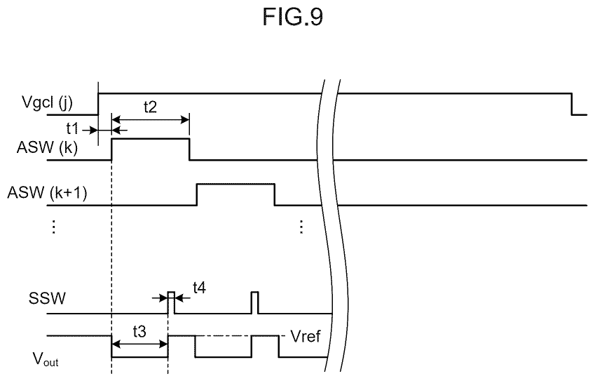

The following describes an operation example of the detection device 1. FIG. 6 is a timing waveform diagram illustrating an operation example of the detection device. FIG. 7 is a timing waveform diagram illustrating an operation example in the reset period in FIG. 6. FIG. 8 is a timing waveform diagram illustrating an operation example in the readout period in FIG. 6. FIG. 9 is a timing waveform diagram illustrating an operation example in a drive period of one gate line included in a readout period VR in FIG. 6. FIG. 10 is an explanatory diagram for explaining a relation between driving of the sensor area and lighting operations of light sources in the detection device.

As illustrated in FIG. 6, the detection device 1 has the reset period Prst, an exposure period Pex, and the readout period Pdet. The power supply circuit 123 supplies the sensor power supply signal VDDSNS to the anode of the optical sensor PD over the reset period Prst, the exposure period Pex, and the readout period Pdet. The sensor power supply signal VDDSNS is a signal that applies a reverse bias between the anode and the cathode of the optical sensor PD. For example, the reference signal COM of substantially 0.75 V is applied to the cathode of the optical sensor PD, and the sensor power supply signal VDDSNS of substantially −1.25 V is applied to the anode thereof. As a result, a reverse bias of substantially 2.0 V is applied between the anode and the cathode. The control circuit 122 sets the reset signal RST2 to “H”, and then, supplies the start signal STV and the clock signal CK to the gate line drive circuit 15 to start the reset period Prst. During the reset period Prst, the control circuit 122 supplies the reference signal COM to the reset circuit 17, and uses the reset signal RST2 to turn on the fourth switching elements TrR for supplying a reset voltage. This operation supplies the reference signal COM as the reset voltage to each of the signal lines SGL. The reference signal COM is set to 0.75 V, for example.

During the reset period Prst, the gate line drive circuit 15 sequentially selects the gate lines GCL based on the start signal STV, the clock signal CK, and the reset signal RST1. The gate line drive circuit 15 sequentially supplies gate drive signals Vgcl{Vgcl(1), . . . , Vgcl(M)} to the gate lines GCL. Each of the gate drive signals Vgcl has a pulsed waveform having a power supply voltage VDD serving as a high-level voltage and a power supply voltage VSS serving as a low-level voltage. In FIG. 6, M gate lines GCL are provided (where M is, for example, 256), and the gate drive signals Vgcl(1), . . . , Vgcl(M) are sequentially supplied to the respective gate lines GCL. Thus, the first switching elements Tr are sequentially brought into a conducting state and supplied with the reset voltage on a row-by-row basis. For example, a voltage of 0.75 V of the reference signal COM is supplied as the reset voltage.

Specifically, as illustrated in FIG. 7, the gate line drive circuit 15 supplies the gate drive signal Vgcl(1) at the high-level voltage (power supply voltage VDD) to the gate line GCL(1) during a period V(1). The control circuit 122 supplies any one of selection signals ASW1, . . . , ASW6 (selection signal ASW1 in FIG. 7) to the signal line selection circuit 16 during a period in which the gate drive signal Vgcl(1) is at the high-level voltage (power supply voltage VDD). This operation couples the signal line SGL selected by any one of the selection signals ASW1, . . . , ASW6 (selection signal ASW1 in FIG. 7) to the AFE circuit 48. As a result, the reset voltage (reference signal COM) is also supplied to coupling wiring between the third switching element TrS and the AFE circuit 48.

In the same way, the gate line drive circuit 15 supplies the gate drive signals Vgcl(2), . . . , Vgcl(M−1), Vgcl(M) at the high-level voltage to gate lines GCL(2), . . . , GCL(M−1), GCL(M) during periods V(2), . . . , V(M−1), V(M), respectively.

Thus, during the reset period Prst, the capacitive elements Ca and the optical sensors PD of all the partial detection areas PAA are sequentially electrically coupled to the signal lines SGL, and are supplied with the reference signal COM. As a result, the voltage between opposite ends of the capacitance of the capacitive elements Ca is reset. The voltage between opposite ends of the capacitance of the capacitive elements Ca of some of the partial detection areas PAA can be reset by partially selecting the gate lines and the signal lines SGL.

Examples of the method of controlling the exposure timing include, but are not limited to, a method of controlling the exposure during non-selection of the gate lines and a method of always controlling the exposure. In the method of controlling the exposure during non-selection of the gate lines, the gate drive signals {Vgcl(1), . . . , Vgcl(M)} are sequentially supplied to all the gate lines GCL coupled to the optical sensors PD serving as the detection targets, and all the optical sensors PD serving as the detection targets are supplied with the reset voltage. Then, after all the gate lines GCL coupled to the optical sensors PD serving as the detection targets are set to a low voltage (the first switching elements Tr are turned off), the exposure starts and the exposure is performed during the exposure period Pex. After the exposure ends, the gate drive signals {Vgcl(1), . . . , Vgcl(M)} are sequentially supplied to the gate lines GCL coupled to the optical sensors PD serving as the detection targets as described above, and reading out is performed during the readout period Pdet. In the method of always controlling the exposure, the control for performing the exposure can also be performed during the reset period Prst and the readout period Pdet (the exposure is always controlled). In this case, the substantial exposure period Pex(1) starts immediately after the gate drive signal Vgcl(1) supplied to the gate line GCL changes from L (VSS) to H (VDD) in the reset period Prst. The substantial exposure periods Pex{(1), . . . , (M)} are periods during which the capacitive elements Ca are charged from the optical sensors PD by an effect of light or the capacitive elements Ca are discharged to the optical sensors PD. The electric charge stored in the capacitive elements Ca during the reset period Prst causes a reverse directional current to flow (from the cathode to the anode) through the optical sensor PD due to light irradiation, and the potential difference in the capacitive element Ca decreases. The start timing and the end timing of the substantial exposure periods Pex(1), . . . , Pex(M) are different among the partial detection areas PAA corresponding to the gate lines GCL. Each of the substantial exposure periods Pex(1), . . . , Pex(M) starts when the gate drive signal Vgcl changes from the power supply voltage VDD serving as the high-level voltage to the power supply voltage VSS serving as the low-level voltage during the reset period Prst. Each of the substantial exposure periods Pex(1), . . . , Pex(M) ends when the gate drive signal Vgcl changes from the power supply voltage VSS to the power supply voltage VDD during the readout period Pdet. The lengths of the exposure time of the substantial exposure periods Pex(1), . . . , Pex(M) are equal.

During the substantial exposure periods Pex {(1), . . . , (M)}, a current flows correspondingly to the light irradiating the optical sensor PD in each of the partial detection areas PAA. As a result, an electric charge is stored in each of the capacitive elements Ca, or an electric charge is discharged from each of the capacitive elements Ca.

At a time before the readout period Pdet starts, the control circuit 122 sets the reset signal RST2 to a low-level voltage. This operation stops the operation of the reset circuit 17. The reset signal may be set to a high-level voltage only during the reset period Prst. During the readout period Pdet, the gate line drive circuit 15 sequentially supplies the gate drive signals Vgcl(1), . . . , Vgcl(M) to the gate lines GCL in the same way as that during the reset period Prst.

Specifically, as illustrated in FIG. 8, the gate line drive circuit 15 supplies the gate drive signal Vgcl(1) at the high-level voltage (power supply voltage VDD) to the gate line GCL(1) during a row readout period VR(1). The control circuit 122 sequentially supplies the selection signals ASW1, . . . , ASW6 to the signal line selection circuit 16 while the gate drive signal Vgcl(1) is at the high-level voltage (power supply voltage VDD). This operation sequentially or simultaneously couples the signal lines SGL of the partial detection areas PAA selected by the gate drive signal Vgcl(1) to the AFE circuit 48. As a result, the detection signal Vdet for each of the partial detection areas PAA is supplied to the AFE circuit 48.

In the same way, the gate line drive circuit 15 supplies the gate drive signals Vgcl(2), . . . , Vgcl(M−1), Vgcl(M) at the high-level voltage to the gate lines GCL(2), . . . , GCL(M−1), GCL(M) during row readout periods VR(2), . . . , VR(M−1), VR(M), respectively. That is, the gate line drive circuit 15 supplies the gate drive signal Vgcl to the gate line GCL in each of the row readout periods VR(1), VR(2), . . . , VR(M−1), VR(M). The signal line selection circuit 16 sequentially selects the signal lines SGL based on the selection signal ASW during each period in which the gate drive signal Vgcl is set to the high-level voltage. The signal line selection circuit 16 sequentially couples each of the signal lines SGL to one AFE circuit 48. Thus, the detection device 1 can output the detection signals Vdet of all the partial detection areas PAA to the AFE circuit 48 during the readout period Pdet.

With reference to FIG. 9, the following describes an operation example during the row readout period VR that is a supply period of one gate drive signal Vgcl(j) in FIG. 6. In FIG. 6, the reference numeral of the row readout period VR is assigned to the first gate drive signal Vgcl(1). The same also applies to the other gate drive signals Vgcl(2), . . . , Vgcl(M). j is any one of the natural numbers 1 to M.

As illustrated in FIGS. 9 and 4, an output (Vout) of each of the third switching elements TrS has been reset to the reference potential (Vref) voltage in advance. The reference potential (Vref) voltage serves as the reset voltage, and is set to 0.75 V, for example. Then, the gate drive signal Vgcl(j) is set to a high level, and the first switching elements Tr of a corresponding row are turned on. Thus, each of the signal lines SGL in each row is set to a voltage corresponding to the electric charge stored in the capacitance (capacitive element Ca) of the partial detection area PAA. After a period t1 elapses from a rising edge of the gate drive signal Vgcl(j), a period t2 starts in which the selection signal ASW(k) is set to a high level. When the selection signal ASW(k) is set to the high level and the third switching element TrS is turned on, by the electric charge that has been charged in the capacitance (capacitive element Ca) of the partial detection area PAA electrically coupled to the AFE circuit 48 via the third switching element TrS, the output (Vout) of the third switching element TrS (refer to FIG. 4) is changed to a voltage corresponding to the electric charge stored in the capacitance (capacitive element Ca) of the partial detection area PAA (period t3). In the example of FIG. 9, this voltage is reduced from the reset voltage as illustrated in the period t3. Then, when the switch SSW is on (period t4 during which an SSW signal is set to a high level), the electric charge stored in the capacitance (capacitive element Ca) of the partial detection area PAA is transferred to the capacitance (capacitive element Cb) of the detection signal amplifying circuit 42 of the AFE circuit 48, and the output voltage of the detection signal amplifying circuit 42 is set to a voltage corresponding to the electric charge stored in the capacitive element Cb. At this time, the potential of the inverting input part of the detection signal amplifying circuit 42 is a virtual short-circuit potential of an operational amplifier, and therefore, set to the reference potential (Vref). The A/D conversion circuit 43 reads out the output voltage of the detection signal amplifying circuit 42. In the example of FIG. 9, the waveforms of the selection signals ASW(k), ASW(k+1), . . . corresponding to the signal lines SGL of the respective columns are set high to sequentially turn on the third switching elements TrS, and the same operation is sequentially performed to sequentially read out the electric charges stored in the capacitance (capacitive elements Ca) of the partial detection areas PAA coupled to the gate line GCL. ASW(k), ASW(k+1), . . . in FIG. 9 are, for example, any of ASW1 to ASW6 in FIG. 9.

Specifically, after the period t4 starts in which the switch SSW is on, the electric charge is transferred from the capacitance (capacitive element Ca) of the partial detection area PAA to the capacitance (capacitive element Cb) of the detection signal amplifying circuit 42 of the AFE circuit 48. At this time, the non-inverting input (+) of the detection signal amplifying circuit 42 is set to the reference potential (Vref) voltage (at 0.75 V, for example). Therefore, the output (Vout) of the third switching element TrS is also set to the reference potential (Vref) voltage due to the virtual short-circuit between the inputs of the detection signal amplifying circuit 42. The voltage of the capacitive element Cb is set to a voltage corresponding to the electric charge stored in the capacitance (capacitive element Ca) of the partial detection area PAA at a location where the third switching element TrS is turned on in response to the selection signal ASW(k). After the output (Vout) of the third switching element TrS is set to the reference potential (Vref) voltage due to the virtual short-circuit, the output of the detection signal amplifying circuit 42 reaches a voltage corresponding to the capacitance of the capacitive element Cb, and this output voltage is read by the A/D conversion circuit 43. The voltage of the capacitive element Cb is, for example, a voltage between two electrodes provided on a capacitor constituting the capacitive element Cb. The reset switch RSW is turned off after having been turned on from an off state before the switch SSW is turned on. As a result, the initial value of the voltage between opposite ends of the capacitive element Cb is substantially 0 V.

The period t1 is, 20 μs, for example. The period t2 is, 60 μs, for example. The period t3 is, 44.7 μs, for example. The period t4 is, 0.98 μs, for example.

As illustrated in FIG. 10, the detection device 1 executes the reset period Prst, the exposure periods Pex {(1), . . . , (M)}, and the readout period Pdet described above in each of the periods t(1), t(2), t(3), and t(4). In the reset period Prst and the readout period Pdet, the gate line drive circuit 15 sequentially scans the gate lines from GCL(1) to GCL(M). In the following description, the term “one-frame detection” denotes the detection in each period t, that is, the detection in which the gate lines are scanned from GCL(1) to GCL(M) in the reset period Prst and the readout period Pdet and the detection signals Vdet are acquired from the signal lines SGL in the respective columns.

The control circuit 122 can control the lighting and the non-lighting of the light sources depending on the detection target. FIG. 10 illustrates an example in which the first light sources 61 are turned on during the periods t(1) and t(3), and the second light sources 62 are turned on during the periods t(2) and t(4). That is, in an example illustrated in FIG. 10, the control circuit 122 alternately turns on and off the first light sources 61 and the second light sources 62 for each one-frame detection. The present disclosure is not limited to this example. For example, the control circuit 122 may turn on and off the first light sources 61 and the second light sources 62 at intervals of a predetermined period of time, or may successively turn on either of the first and the second light sources 61 and 62.

FIGS. 6 to 10 illustrate the example in which the gate line drive circuit 15 individually selects the gate line GCL, but the present disclosure is not limited to this example. The gate line drive circuit 15 may simultaneously select a predetermined number (two or more) of the gate lines GCL, and sequentially supply the gate drive signals Vgcl to the gate lines GCL in units of the predetermined number of the gate lines GCL. The signal line selection circuit 16 may also simultaneously couple a predetermined number (two or more) of the signal lines SGL to one AFE circuit 48. Moreover, the gate line drive circuit 15 may scan some of the gate lines GCL while skipping the others.

As illustrated in FIG. 8, in the row readout period VR(1), the selection signals ASW1, . . . , ASW6 are sequentially supplied to the signal line selection circuit 16 during the period in which the gate drive signal Vgcl(1) is at the high-level voltage (power supply voltage VDD).

As described above, the detection device 1 has the configuration including, for example, a plurality of types of light sources (first light sources 61 and second light sources 62) that emit light having different wave lengths, and thereby, can acquire the fingerprint acquired by detecting the light reflected on the surface of the finger of the subject and the various types of biometric information acquired by detecting the light reflected in or transmitted through the finger or the wrist of the subject.

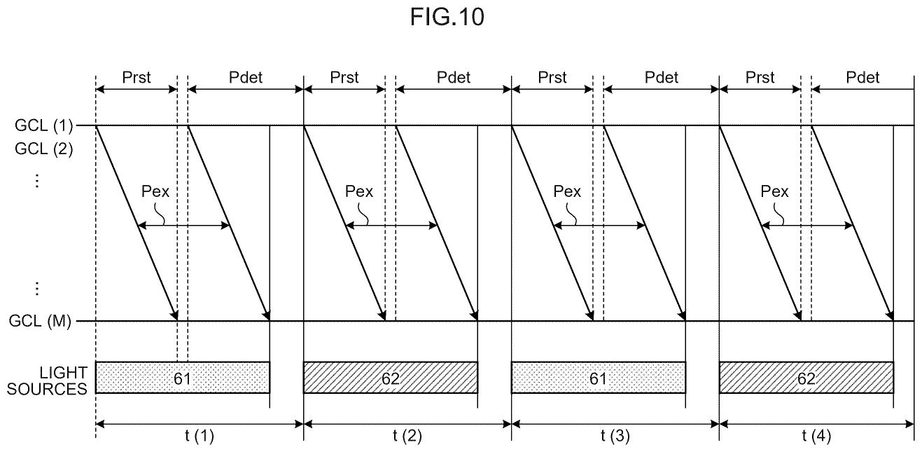

As a specific example of the information on the living body acquired by the detection device 1, the following describes an example of acquiring the pulse waves serving as the biometric information for calculating the oxygen saturation level in blood (hereinafter, called “blood oxygen saturation level (SpO₂)”). FIG. 11 is a plan view schematically illustrating a relation between the sensor area, the first light sources, and the second light sources in the detection device according to the embodiment.

As illustrated in FIG. 11, the detection device 1 includes a filter 63. The filter 63 is disposed so as to overlap the detection area AA from one end to the other end of the sensor area 10 in a scan direction SCAN. The filter 63 has a transmission bandwidth for transmitting the first light emitted from the first light sources 61 and the second light emitted from the second light sources 62. In the configuration according to the first embodiment, the filter 63 is not required, and the configuration may exclude the filter 63.

In the configuration illustrated in FIG. 11, the scan direction SCAN is a direction in which the gate line drive circuit 15 scans the gate line GCL. That is, one gate line GCL is provided so as to extend in the first direction Dx in the detection area AA and is coupled to the partial detection areas PAA provided in the detection area AA. One signal line SGL is provided so as to extend in the second direction Dy in the detection area AA and is coupled to the optical sensors PD in the detection area AA.

The first light source base member 51 and the second light source base member 52 face each other in the first direction Dx with the detection area AA interposed therebetween in plan view. The first and the second light sources 61 and 62 are provided on a surface of the first light source base member 51 facing the second light source base member 52. The first and the second light sources 61 and 62 are also provided on a surface of the second light source base member 52 facing the first light source base member 51. The first and the second light sources 61 and 62 are arranged in the first direction Dx along the periphery of the detection area AA and are alternately provided in the second direction Dy on each of the first light source base member 51 and the second light source base member 52.

The first light sources 61 emit the first light in a direction parallel to the first direction Dx. As a result, the detection area AA is irradiated with the first light. The second light sources 62 emit the second light in the direction parallel to the first direction Dx. As a result, the detection area AA is irradiated with the second light.

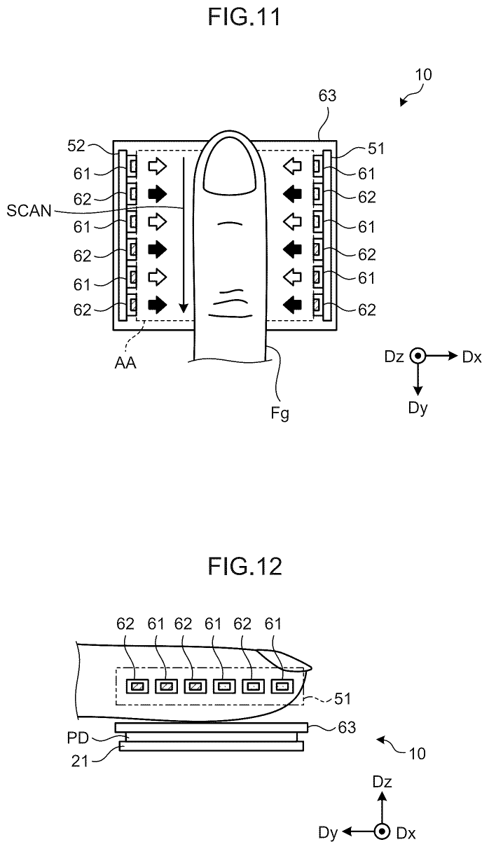

FIG. 12 is a side view of the detection device illustrated in FIG. 11 as viewed in the first direction Dx. As illustrated in FIG. 12, an object to be detected such as the finger Fg or the wrist of the subject comes in contact with or in proximity to the top of the sensor area 10 with the filter 63 interposed therebetween. The first and the second light sources 61 and 62 are arranged above the sensor area 10 and the filter 63 and are arranged with the object to be detected such as the finger Fg or the wrist of the subject interposed therebetween in the first direction Dx.

In this example, for example, red visible light (red light) having a wavelength of 600 nm to 700 nm, specifically approximately 660 nm is employed as the first light emitted from the first light sources 61, and infrared light having a wavelength of 780 nm to 950 nm, specifically approximately 850 nm is employed as the second light emitted from the second light sources 62. When acquiring the human blood oxygen saturation level (SpO₂), a pulse wave acquired using the first light (red light) and a pulse wave acquired using the second light (infrared light) are used. Alternatively, the green light of 420 nm to 600 nm, for example, approximately 500 nm may be employed as the second light emitted from the second light sources 62.

Since the amount of light absorbed changes depending on the amount of oxygen taken in by hemoglobin, the optical sensor PD detects the amount of light obtained by subtracting the light absorbed by blood (hemoglobin) from the irradiating first and second light. Most of the oxygen in blood is reversibly bound to hemoglobin in red blood cells, and a small portion is dissolved in plasma. More specifically, the value of what percentage of a permissible amount of oxygen is bound to blood as a whole is called the oxygen saturation level (SpO₂). At the two wavelengths of the first light and the second light, the blood oxygen saturation level can be calculated from the amount obtained by subtracting the amount of the light absorbed by blood (hemoglobin) from the amount of the emission light.

The blood oxygen saturation level (SpO₂) is determined by the ratio of hemoglobin in blood bound to oxygen (oxygenated hemoglobin (O2Hb)) to hemoglobin in blood not bound to oxygen (reduced hemoglobin (HHb)). The light absorption characteristics of the red light are represented as HHb >>O2Hb, indicating that HHb has significantly higher absorbance, while the light absorption characteristics of the infrared light are represented as HHb≈O2Hb, indicating that O2Hb has slightly higher absorbance.

The first light emitted from the first light sources 61 travels in the direction parallel to the first direction Dx and enters the finger Fg or the wrist of the subject. The first light emitted from the first light sources 61 penetrates into the living body and is reflected in the finger Fg or the wrist of the subject. The reflected light reflected in the finger Fg or the wrist of the subject travels in the third direction Dz and enters the detection area AA of the sensor area 10 through the filter 63.

The second light emitted from the second light sources 62 travels in the direction parallel to the first direction Dx and enters the finger Fg or the wrist of the subject. The second light emitted from the second light sources 62 penetrates into the living body and is reflected in the finger Fg or the wrist of the subject. The reflected light reflected in the finger Fg or the wrist of the subject travels in the third direction Dz and enters the detection area AA of the sensor area 10 through the filter 63.

The arrangement of the first and the second light sources 61 and 62 is not limited to the example illustrated in FIGS. 11 and 12. For example, the first and the second light may be emitted from above the object to be detected such as the finger Fg or the wrist of the subject illustrated in FIG. 12, specifically, in the third direction Dz. Alternatively, the first and the second light sources 61 and 62 may be, for example, what are called direct-type light sources provided directly below the detection area AA.

In the example illustrated in FIG. 10, the reset period Prst, the exposure period Pex, and the readout period Pdet are provided for the one-frame detection in each of the periods t(1), t(2), t(3), and t(4). In the reset period Prst and the readout period Pdet, the gate line drive circuit 15 sequentially scans the gate lines from GCL(1) to GCL(M).

As illustrated in FIG. 10, in the detection for one frame in the period t(1), the control circuit 122 (detection control circuit 11) causes the first light sources 61 to be on and the second light sources 62 to be off during the exposure period Pex. In the detection for one frame in the period t(2), the control circuit 122 (detection control circuit 11) causes the first light sources 61 to be off and the second light sources 62 to be on during the exposure period Pex. In the same way, the first light sources 61 are on and the second light sources 62 are off during the exposure period Pex in the detection for one frame in the period t(3), and the first light sources 61 are off and the second light sources 62 are on during the exposure period Pex in the detection for one frame in the period t(4).

Thus, the first and the second light sources 61 and 62 are controlled to be on and off in a time-division manner for each detection operation for one frame. As a result, a first detection value detected by the optical sensor PD using the first light and a second detection value detected by the optical sensor PD using the second light are output to the AFE circuit 48 in a time-division manner.

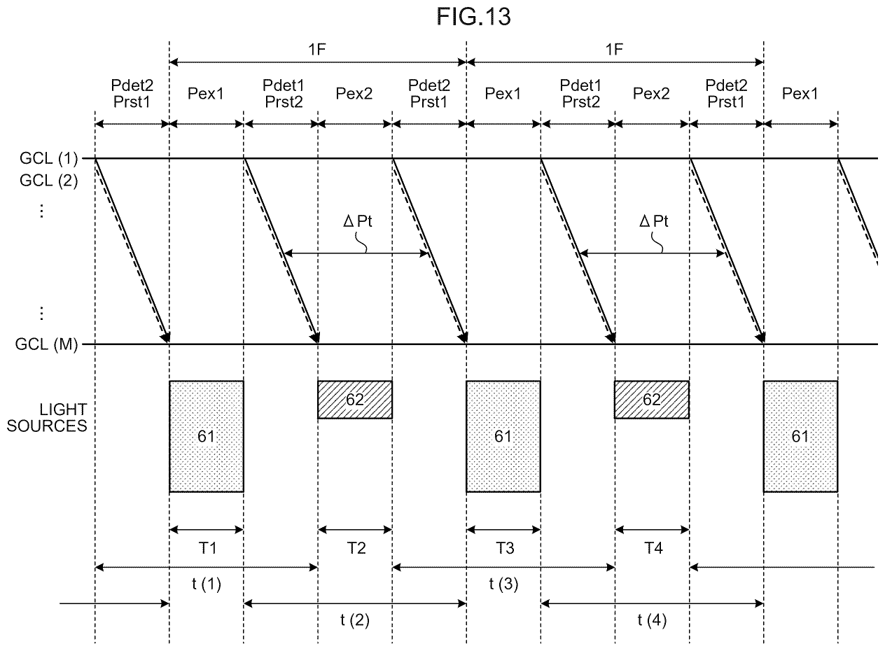

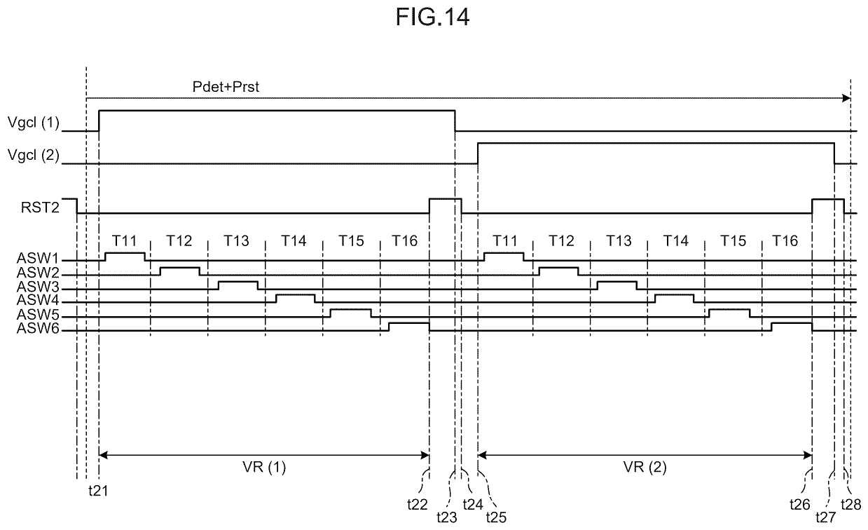

In calculating the blood oxygen saturation level (SpO₂), the pulse wave acquired using the first light and the pulse wave acquired using the second light are used. Therefore, the difference in detection timing between the first detection value detected using the first light and the second detection value detected using the second light is preferably smaller. The following describes an operation example that can reduce the difference in detection timing between the first detection value detected using the first light and the second detection value detected using the second light, with reference to FIGS. 13 and 14.

FIG. 13 is an explanatory diagram for explaining an operation example of the detection device according to a first technical example according to the first embodiment. FIG. 14 is a timing waveform diagram illustrating the operation example of the detection device according to the first technical example according to the first embodiment.

In the first technical example according to the first embodiment illustrated in FIG. 13, the first light is the red light and the second light is the infrared light. In the first technical example according to the first embodiment illustrated in FIG. 13, a solid-line arrow indicates a first reset period Prst1 in the detection operation using the first light and a second reset period Prst2 in the detection operation using the second light, and a dashed-line arrow indicates a first readout period Pdet1 in the detection operation using the first light and a second readout period Pdet2 in the detection operation using the second light.

In the first technical example according to the first embodiment illustrated in FIG. 13, the size in the height direction of each light emission period conceptually illustrates an emission intensity of each group of the first light sources 61 and the second light sources 62. The red light serving as the first light is absorbed more by a human body than the infrared light serving as the second light. For this reason, in the first technical example according to the first embodiment illustrated in FIG. 13, the emission intensity of the first light is set to be higher than that of the second light.

In the first technical example according to the first embodiment illustrated in FIG. 13, the detection operation using the first light is performed during the periods t(1), t(3), . . . , and the detection operation using the second light is performed during the periods t(2), t(4), . . . . Hereinafter, the periods t(1), t(3), . . . during which the detection operation based on the first light is performed are each also referred to as a “first light detection period”, and the periods t(2), t(4), . . . during which the detection operation based on the second light is performed are each also referred to as a “second light detection period”. The first detection value and the second detection value used to calculate the blood oxygen saturation level (SpO₂) are detected for each frame (1F) unit, which is a set of a first exposure period Pex1 of the first light detection period, the first readout period Pdet1 of the first light detection period, a second exposure period Pex2 of the second light detection period, and the second readout period Pdet2 of the second light detection period. In the first technical example according to the first embodiment illustrated in FIGS. 13 and 14, the light emission period of the first light sources 61 almost coincides with the first exposure period Pex1 in the first light detection period. In the first technical example according to the first embodiment illustrated in FIGS. 13 and 14, the light emission period of the second light sources 62 almost coincides with the second exposure period Pex2 in the second light detection period.

In the first technical example according to the first embodiment illustrated in FIG. 13, the first reset period Prst1 in the first light detection period and the second readout period Pdet2 in the second light detection period of the previous frame are executed in parallel. In the one frame (1F), the second reset period Prst2 in the second light detection period and the first readout period Pdet1 in the first light detection period are executed in parallel. This way of operation can reduce a difference ΔPt in detection timing between the first and the second detection values used to calculate the blood oxygen saturation level (SpO₂).

In the first technical example according to the first embodiment illustrated in FIG. 13, the gate drive signal Vgcl is supplied to the gate line GCL for each row, and the first switching elements Tr belonging to a given row are brought into a coupled state. Specifically, as illustrated in FIG. 14, at time t21, the gate line drive circuit 15 supplies the gate drive signal Vgcl(1) at the high-level voltage (power supply voltage VDD) to the gate line GCL(1). The row readout period VR(1) starts at time t21 when the gate drive signal Vgcl(1) becomes the high-level voltage.

Specifically, the control circuit 122 sequentially supplies the selection signals ASW1, . . . , ASW6 to the signal line selection circuit 16 during the period in which the gate drive signal Vgcl(1) is at the high-level voltage (power supply voltage VDD). The third switching elements TrS are sequentially brought into the coupled state in response to the selection signals ASW1, . . . , ASW6. That is, during the period of reading out each row (row readout period VR(1)), when the first switching elements Tr of the given row are in the coupled state, the signal line selection circuit 16 couples the signal lines SGL to the AFE circuit 48 column by column in a predetermined order. As a result, the detection signal Vdet for each of the partial detection areas PAA is supplied to the AFE circuit 48.

In FIG. 14, the selection signals ASW1, . . . , ASW6 are supplied in the order of periods T11, . . . , T16 in a time-division manner. At time t22, the control circuit 122 sets the selection signal ASW6 to the low-level voltage, and the reading out of the last column ends. That is, the row readout period VR(1) ends when the gate drive signal Vgcl(1) is at the high-level voltage and the selection signal ASW6 has changed to the low-level voltage.

After the completion of the readout period of the given row (row readout period VR(1)) and before the start of the readout period of a row next to the given row (row readout period VR(2)), a reset potential (reference signal COM) is supplied to the optical sensors PD and the signal lines SGL belonging to the given row. Specifically, the control circuit 122 supplies the reset signal RST2 to the reset signal line Lrst at time t22. This operation turns on the fourth switching elements TrR to supply the reference signal COM to the optical sensors PD and the signal lines SGL corresponding to the gate line GCL(1).

In the example illustrated in FIG. 14, the time when the reset signal RST2 is set to the high-level voltage coincides with the time when the selection signal ASW6 is set to the low-level voltage, at time t22. However, the timing is not limited thereto. The reset signal RST2 may be set to the high-level voltage after a predetermined period of time has elapsed since the selection signal ASW6 has been set to the low-level voltage.

Then, at time t23, the gate line drive circuit 15 sets the gate drive signal Vgcl(1) to the low-level voltage. This operation brings the first switching elements Tr of the certain row into a non-coupled state. At time t24, the control circuit 122 sets the reset signal RST2 to the low-level voltage. This operation ends the readout period Pdet and the reset period Prst of the first row. The capacitance Cb of the AFE circuit 48 is reset by setting the reset switch RSW from the off state via the on state to the off state between time t22 and t24.

Then, at time t25, the gate line drive circuit 15 supplies the gate drive signal Vgcl(2) at the high-level voltage (power supply voltage VDD) to the gate line GCL(2) of the second row. Subsequently, in the same way as in the first row, the readout period Pdet and the reset period Prst of the second row are provided from time t26 to time t28. The detection for one frame (1F) can be performed by repeating the scanning operation described above to the last row (gate line GCL(256)).

In the first technical example according to the first embodiment illustrated in FIGS. 13 and 14, the first reset period Prst1 in the first light detection period (t(1), t(3), . . . ) and the second readout period Pdet2 in the second light detection period of the previous frame are executed in parallel, as described above. The second reset period Prst2 in the second light detection period (t(2), t(4), . . . ) and the first readout period Pdet1 in the first light detection period are executed in parallel. This way of operation can reduce the difference ΔPt in detection timing between the first and the second detection values used to calculate the blood oxygen saturation level (SpO₂). The first detection value and the second detection value used to calculate the blood oxygen saturation level (SpO₂) are detected for each frame (1F) unit, which is a set of the first exposure period Pex1 of the first light detection period, the first readout period Pdet1 of the first light detection period, the second exposure period Pex2 of the second light detection period, and the second readout period Pdet2 of the second light detection period.

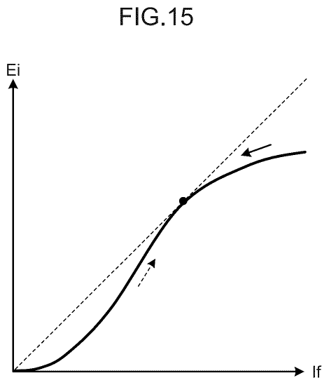

FIG. 15 is a conceptual diagram illustrating a relation between an amount of current flowing in a light source and an emission intensity thereof. In FIG. 15, the horizontal axis indicates a current If flowing through the light source, and the vertical axis indicates an emission intensity Ei of the light source. A dashed line illustrated in FIG. 15 indicates ideal characteristics of the luminous efficiency of the light source.

As described above, the red light serving as the first light is absorbed more by the human body than the infrared light serving as the second light. In the first technical example according to the first embodiment illustrated in FIG. 13, the emission intensity of the first light is set to be higher than that of the second light, but in the present embodiment, the emission intensity of the red light serving as the first light is reduced and the light emission period is relatively lengthened. As a result, the luminous efficiency of the first light sources 61 can be optimized, for example, as indicated by a solid-line arrow in FIG. 15. Alternatively, the emission intensity of the infrared light serving as the second light is increased and the light emission period is relatively shortened. As a result, the luminous efficiency of the second light sources 62 can be optimized, for example, as indicated by a dashed-line arrow in FIG. 15.

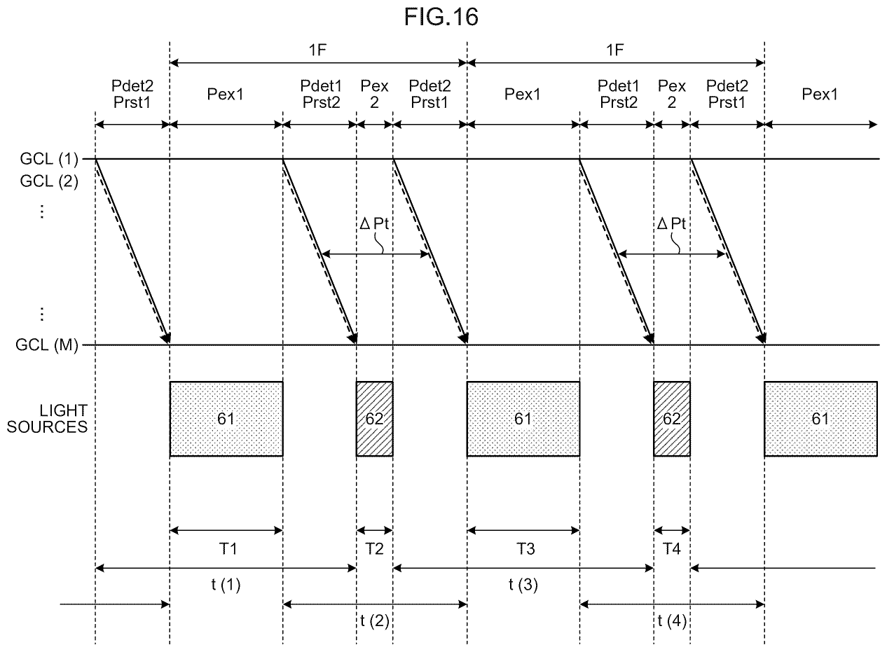

FIG. 16 is an explanatory diagram for explaining an operation example of the detection device according to the first embodiment. In the operation example of the detection device 1 according to the first embodiment illustrated in FIG. 16, the light emission period of the first light sources 61, that is, the first exposure period Pex1 in the first light detection period, is longer and the light emission period of the second light sources 62, that is, the second exposure period Pex2 in the second light detection period, is shorter than in the first technical example according to the first embodiment illustrated in FIG. 13. In the operation example of the detection device 1 according to the first embodiment illustrated in FIG. 16, the emission intensity of the first light sources 61 is reduced and the emission intensity of the second light sources 62 is increased, relative to the first technical example according to the first embodiment illustrated in FIG. 13.

As a result, the time interval between the first readout period Pdet1 of the first light detection period and the second readout period Pdet2 of the second light detection period can be made shorter than in the first technical example according to the first embodiment illustrated in FIG. 13. The first detection value is a detection value acquired during the first light detection period (t(1), t(3), . . . ) illustrated in FIG. 16, and the second detection value is a detection value acquired during the second light detection period (t(2), t(4), . . . ) illustrated in FIG. 16. The blood oxygen saturation level (SpO₂) is calculated using the first detection value acquired during the first light detection period illustrated in FIG. 16 and the second detection value acquired during the second light detection period illustrated in FIG. 16. As a result, the difference ΔPt in detection timing between the first and the second detection values used to calculate the blood oxygen saturation level (SpO₂) can be made smaller than in the first technical example according to the first embodiment illustrated in FIG. 13. By reducing the emission intensity of the first light sources 61 and relatively increasing the emission intensity of the second light sources 62, the luminous efficiencies of the first light sources 61 and the second light sources 62 can be optimized. This optimization enables power saving in the detection operation in the detection device 1 according to the first embodiment.

### Modification

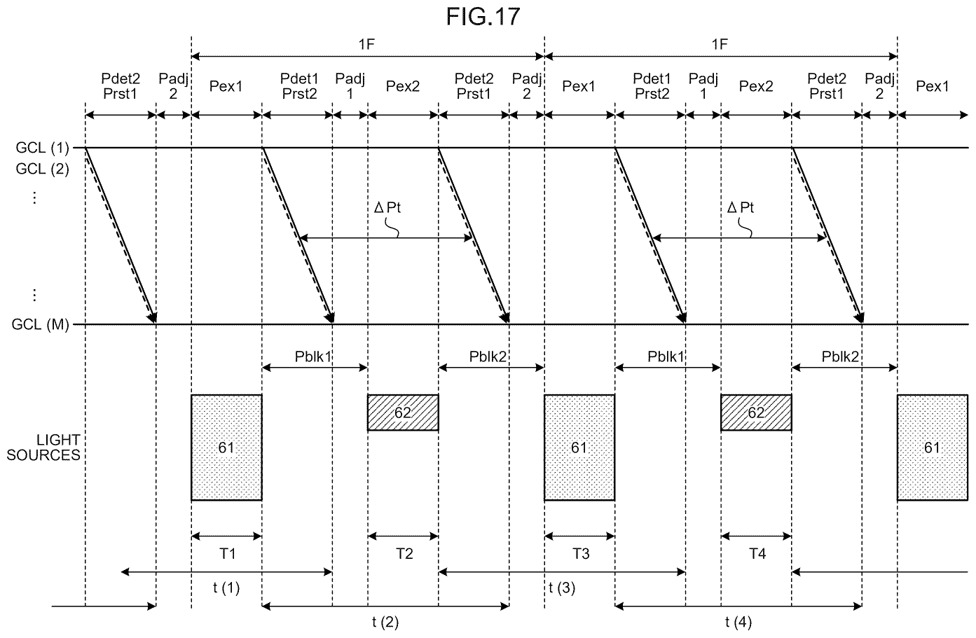

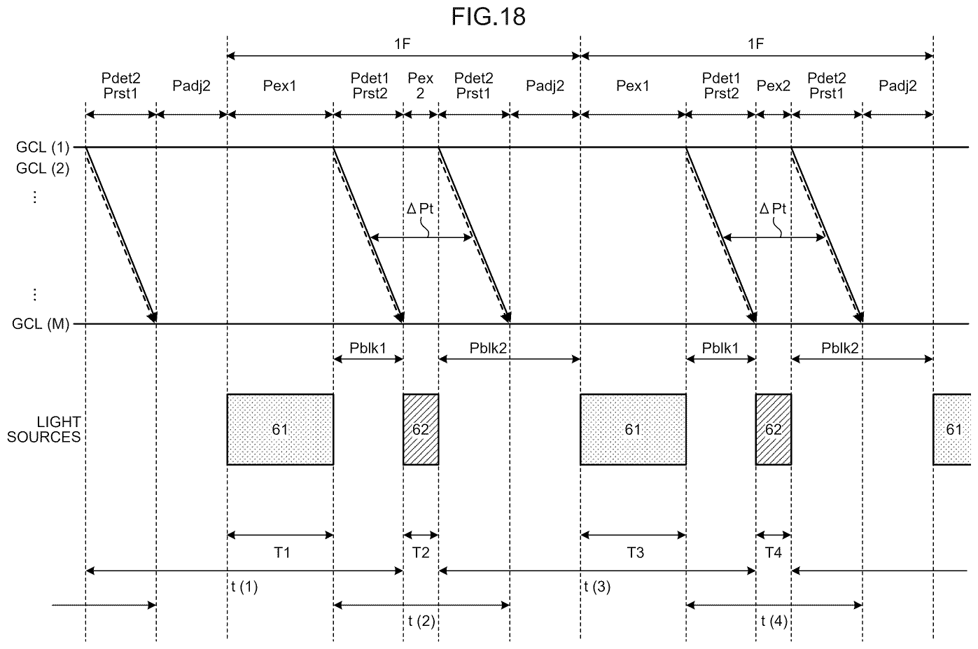

FIG. 17 is an explanatory diagram for explaining an operation example of the detection device according to a second technical example according to the first embodiment. FIG. 18 is an explanatory diagram for explaining an operation example of the detection device according to a modification of the first embodiment. The length of each frame (1F) period is equalized between the second technical example according to the first embodiment illustrated in FIG. 17 and the modification of the first embodiment illustrated in FIG. 18 by providing a frame-period adjustment period to be described later.

In the operation example of the detection device 1 according to the modification of the first embodiment illustrated in FIG. 18, the light emission period of the first light sources 61 is longer and the light emission period of the second light sources 62 is relatively shorter than in the second technical example according to the first embodiment illustrated in FIG. 17, in the same way as in the operation example of the detection device 1 according to the first embodiment illustrated in FIG. 16. In the operating example of the modification of the detection device 1 according to the first embodiment illustrated in FIG. 18, the emission intensity of the first light sources 61 is made lower and the emission intensity of the second light sources 62 is relatively made higher than in the second technical example according to the first embodiment illustrated in FIG. 17.

As a result, the difference ΔPt in detection timing between the first and the second detection values used to calculate the blood oxygen saturation level (SpO₂) can be made smaller than in the second technical example according to the first embodiment illustrated in FIG. 17. The first detection value is a detection value acquired during the first light detection period (t(1), t(3), . . . ) illustrated in FIG. 18, and the second detection value is a detection value acquired during the second light detection period (t(2), t(4), . . . ) illustrated in FIG. 18. The blood oxygen saturation level (SpO₂) is calculated using the first detection value acquired during the first light detection period illustrated in FIG. 18 and the second detection value acquired during the second light detection period illustrated in FIG. 18. By reducing the emission intensity of the first light sources 61 and relatively increasing the emission intensity of the second light sources 62, the detection operation in the modification of the detection device 1 according to the first embodiment can be made more power-saving.

In the second technical example according to the first embodiment illustrated in FIG. 17, after the detection operation using the first light is performed in the periods t(1), t(3), . . . , a first frame-period adjustment period Padj1 is provided between the first readout period Pdet1 and the second exposure period Pex2; and after the detection operation using the second light is performed in the periods t(2), t(4), . . . , a second frame-period adjustment period Padj2 is provided between the second readout period Pdet2 and the first exposure period Pex1. The first frame-period adjustment period Padj1 is a period in the first light detection period that does not substantially contribute to the operation to detect the first light, unlike the first exposure period Pex1 and the first readout period Pdet1. The second frame-period adjustment period Padj2 is a period in the second light detection period that does not substantially contribute to the operation to detect the second light, unlike the second exposure period Pex2 and the second readout period Pdet2. In the present embodiment, a period between the light emission period of the first light sources 61 and the light emission period of the second light sources 62 is defined as a first blanking period Pblk1. In the present embodiment, a period between the light emission period of the second light sources 62 and the light emission period of the first light sources 61 is defined as a second blanking period Pblk2.

In contrast, in the modification of the first embodiment illustrated in FIG. 18, after the detection operation using the first light is performed in the periods t(1), t(3), . . . , the first frame-period adjustment period Padj1 is not provided; whereas, after the detection operation using the second light is performed in the periods t(2), t(4), . . . , the second frame-period adjustment period Padj2 is provided. Thus, the first blanking period Pblk1 does not include the first frame-period adjustment period Padj1, which makes the first blanking period Pblk1 relatively shorter than the second blanking period Pblk2. Therefore, even in the configuration in which the length of the one frame (1F) period is the same as that in the second technical example according to the first embodiment illustrated in FIG. 17, the difference ΔPt in detection timing between the first and the second detection values used to calculate the blood oxygen saturation level (SpO₂) can have the same length as that in the operation example of the detection device 1 according to the first embodiment illustrated in FIG. 16.

Also in the operation example according to the modification of the first embodiment, the first frame-period adjustment period Padj1 can be provided after the detection operation using the first light is performed. In this case, the first frame-period adjustment period Padj1 is made shorter than the second frame-period adjustment period Padj2 (for example, 1/80 the second frame-period adjustment period Padj2 or shorter, preferably 1/100 the second frame-period adjustment period Padj2 or shorter). This configuration can make the difference ΔPt in detection timing between the first and the second detection values used to calculate the blood oxygen saturation level (SpO₂) smaller than in the second technical example according to the first embodiment illustrated in FIG. 17.

### Second Embodiment

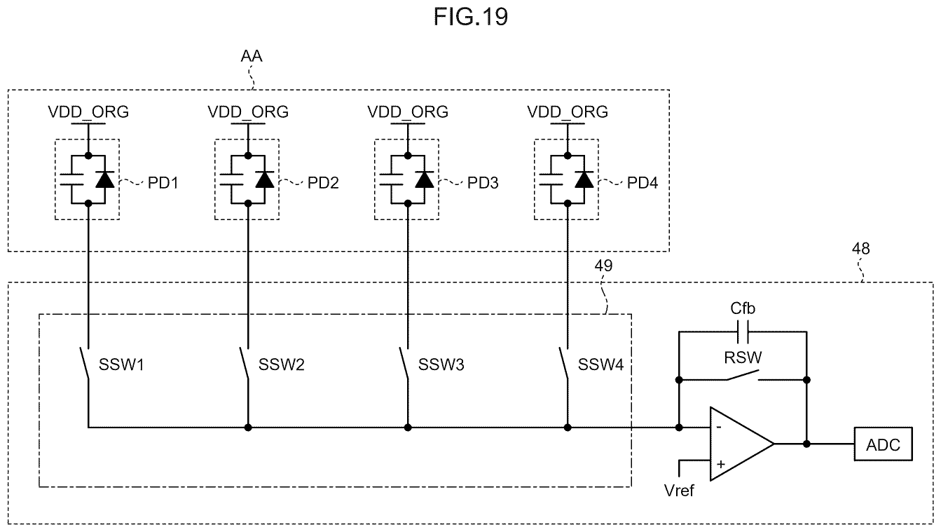

In the first embodiment, the example has been described in which the optical sensors PD are provided in the detection area AA in a matrix having a row-column configuration along the first direction Dx and the second direction Dy. In the second embodiment, a configuration with a smaller number of the optical sensors PD than in the first embodiment will be described. FIG. 19 is a block diagram illustrating an exemplary circuit configuration of the detection device according to a second embodiment of the present disclosure. FIG. 19 illustrates an example in which four optical sensors PD are provided in the detection area AA. The present disclosure is not limited by the arrangement, layout, or the like of optical sensors PD in the detection area AA.

As illustrated in FIG. 19, in the second embodiment, the anodes of optical sensors PD1, PD2, PD3, and PD4 are coupled to the AFE circuit 48 via switches SSW1, SSW2, SSW3, and SSW4, respectively, of a switch circuit 49. The cathodes of optical sensors PD1, PD2, PD3, and PD4 are supplied with a sensor power supply potential VDD ORG from the power supply circuit 123.

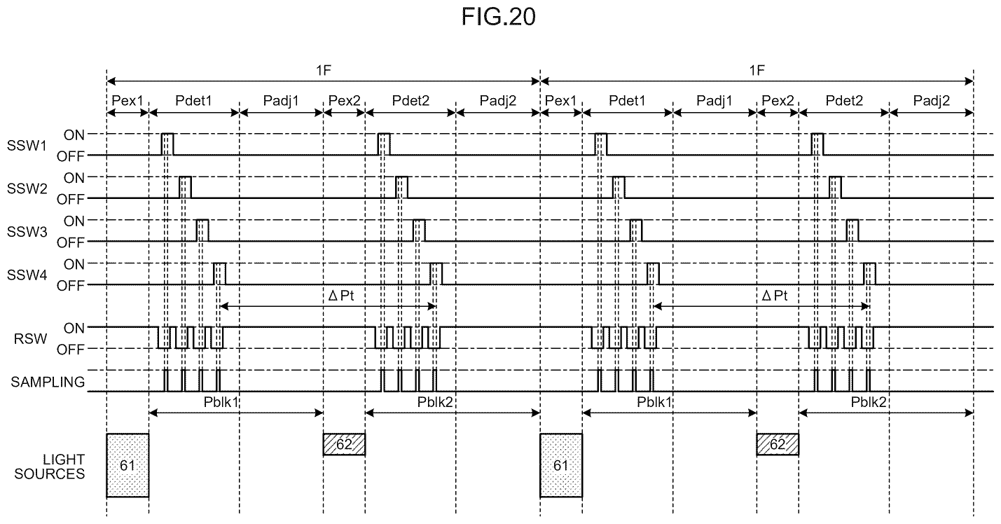

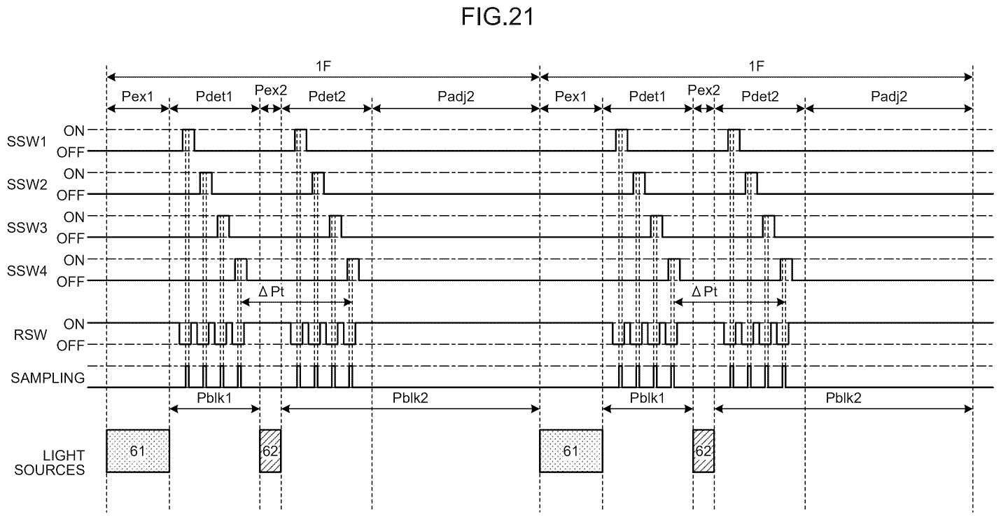

FIG. 20 is an explanatory diagram for explaining an operation example of the detection device according to a comparative example to the second embodiment. FIG. 21 is an explanatory diagram for explaining an operation example of the detection device according to the second embodiment. The length of one frame (1F) period is equalized between the comparative example to the second embodiment illustrated in FIG. 20 and the second embodiment illustrated in FIG. 21 by providing the frame-period adjustment period.

In the operation examples illustrated in FIGS. 20 and 21, in the first readout period Pdet1 after the first exposure period Pex1 of the first light detection period, the switch SSW1 is turned on in an off period of the reset switch RSW, and the first detection value is read out from the optical sensor PD1; then, the reset switch RSW is turned on, and electric charges of the optical sensor PD1 and a capacitive element Cfb are reset; and then, the switch SSW1 is turned off. The switch SSW2 is turned on in the subsequent off period of the reset switch RSW, and the first detection value is read out from the optical sensor PD2; then, the reset switch RSW is turned on, and electric charges of the optical sensor PD2 and the capacitive element Cfb are reset; and then, the switch SSW2 is turned off. The switch SSW3 is turned on in the subsequent off period of the reset switch RSW, and the first detection value is read out from the optical sensor PD3; then, the reset switch RSW is turned on, and electric charges of the optical sensor PD3 and the capacitive element Cfb are reset; and then, the switch SSW3 is turned off. The switch SSW4 is turned on in the subsequent off period of the reset switch RSW, and the first detection value is read out from the optical sensor PD4; then, the reset switch RSW is turned on, and electric charges of the optical sensor PD4 and the capacitive element Cfb are reset; and then, a switch SSW4 is turned off.

Then, in the second readout period Pdet2 after the second exposure period Pex2 of the second light detection period, the switch SSW1 is turned on in the off period of the reset switch RSW, and the second detection value is read out from the optical sensor PD1; then, the reset switch RSW is turned on, and the electric charges of the optical sensor PD1 and the capacitive element Cfb are reset; and then, the switch SSW1 is turned off. The switch SSW2 is turned on in the subsequent off period of the reset switch RSW, and the second detection value is read out from the optical sensor PD2; then, the reset switch RSW is turned on, and the electric charges of the optical sensor PD2 and the capacitive element Cfb are reset; and then, the switch SSW2 is turned off. The switch SSW3 is turned on in the subsequent off period of the reset switch RSW, and the second detection value is read out from the optical sensor PD3; then, the reset switch RSW is turned on, and the electric charges of the optical sensor PD3 and the capacitive element Cfb are reset; and then, the switch SSW3 is turned off. The switch SSW4 is turned on in the subsequent off period of the reset switch RSW, and the second detection value is read out from the optical sensor PD4; then, the reset switch RSW is turned on, and the electric charges of the optical sensor PD4 and the capacitive element Cfb are reset; and then, the switch SSW4 is turned off.

Thus, in the operation examples illustrated in FIGS. 20 and 21, the first detection value and the second detection value used to calculate the blood oxygen saturation level (SpO₂) are detected for each frame (1F) unit, which is a set of the first exposure period Pex1 of the first light detection period, the first readout period Pdet1 of the first light detection period, the second exposure period Pex2 of the second light detection period, and the second readout period Pdet2 of the second light detection period. The first light detection period is a period during which the detection operation using the first light is performed, and the second light detection period is a period during which the detection operation using the second light is performed. The first detection value is the detection value acquired in the first readout period Pdet1 of the first light detection period, and the second detection value is the detection value acquired in the second readout period Pdet2 of the second light detection period. The blood oxygen saturation level (SpO₂) is calculated using the first detection value acquired during the first readout period Pdet1 of the first light detection period and the second detection value acquired during the second readout period Pdet2 of the second light detection period.

In the operation example of the detection device 1 according to the second embodiment illustrated in FIG. 21, the first exposure period Pex1 in the first light detection period is longer and the second exposure period Pex2 in the second light detection period is relatively shorter than in the comparative example to the second embodiment illustrated in FIG. 20. In the operation example of the detection device 1 according to the second embodiment illustrated in FIG. 21, the emission intensity of the first light sources 61 is made lower and the emission intensity of the second light sources 62 is relatively made higher than in the comparative example to the second embodiment illustrated in FIG. 20.

As a result, the time interval between the first readout period Pdet1 of the first light detection period and the second readout period Pdet2 of the second light detection period can be made shorter than in the comparative example illustrated in FIG. 20. Therefore, the difference ΔPt in detection timing between the first and the second detection values used to calculate the blood oxygen saturation level (SpO₂) can be made smaller than in the comparative example illustrated in FIG. 20. By reducing the emission intensity of the first light sources 61 and relatively increasing the emission intensity of the second light sources 62, the luminous efficiencies of the first light sources 61 and the second light sources 62 can be optimized. This optimization enables power saving in the detection operation in the detection device 1 according to the second embodiment.

In the comparative example to the second embodiment illustrated in FIG. 20, after the detection operation using the first light is performed, the first frame-period adjustment period Padj1 is provided between the first readout period Pdet1 and the second exposure period Pex2; and after the detection operation using the second light is performed, the second frame-period adjustment period Padj2 is provided between the second readout period Pdet2 and the first exposure period Pex1. The first frame-period adjustment period Padj1 is a period in the first light detection period that does not substantially contribute to the operation to detect the first light, unlike the first exposure period Pex1 and the first readout period Pdet1. The second frame-period adjustment period Padj2 is a period in the second light detection period that does not substantially contribute to the operation to detect the second light, unlike the second exposure period Pex2 and the second readout period Pdet2. In the present embodiment, the period between the light emission period of the first light sources 61 and the light emission period of the second light sources 62 is defined as the first blanking period Pblk1. In the present embodiment, the period between the light emission period of the second light sources 62 and the light emission period of the first light sources 61 is defined as the second blanking period Pblk2.

The comparative example to the second embodiment illustrated in FIG. 20 illustrates an example where the first blanking period Pblk1 is almost equal to the second blanking period Pblk2.

In contrast, in the operation example of the second embodiment illustrated in FIG. 21, the first frame-period adjustment period Padj1 is not provided after the detection operation using the first light is performed, and the second frame-period adjustment period Padj2 is provided after the detection operation using the second light is performed. Therefore, the first blanking period Pblk1 does not include the first frame-period adjustment period Padj1, which can shorten the first blanking period Pblk1 relative to the second blanking period Pblk2. Therefore, the difference ΔPt in detection timing between the first and the second detection values used to calculate the blood oxygen saturation level (SpO₂) can be made smaller than in the comparative example illustrated in FIG. 20. By reducing the emission intensity of the first light sources 61 and relatively increasing the emission intensity of the second light sources 62, the luminous efficiencies of the first light sources 61 and the second light sources 62 can be optimized. This optimization enables power saving in the detection operation in the detection device 1 according to the second embodiment.

Also in the operation example according to the second embodiment, the first frame-period adjustment period Padj1 can be provided after the detection operation using the first light is performed. In this case, the first frame-period adjustment period Padj1 is made shorter than the second frame-period adjustment period Padj2 (for example, 1/80 the second frame-period adjustment period Padj2 or shorter, preferably 1/100 the second frame-period adjustment period Padj2 or shorter). This configuration can make the difference ΔPt in detection timing between the first and the second detection values used to calculate the blood oxygen saturation level (SpO₂) smaller than in the comparative example illustrated in FIG. 20.

### Third Embodiment

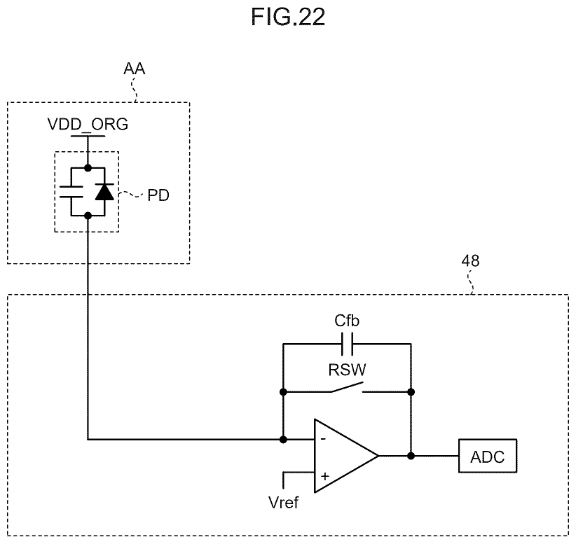

In a third embodiment of the present disclosure, a configuration including one optical sensor PD will be described. FIG. 22 is a block diagram illustrating an exemplary circuit configuration the detection device according to the third embodiment.

As illustrated in FIG. 22, in the third embodiment, the anode of the optical sensor PD is coupled to the AFE circuit 48. The cathode of the optical sensor PD is supplied with the sensor power supply potential VDD ORG from the power supply circuit 123.

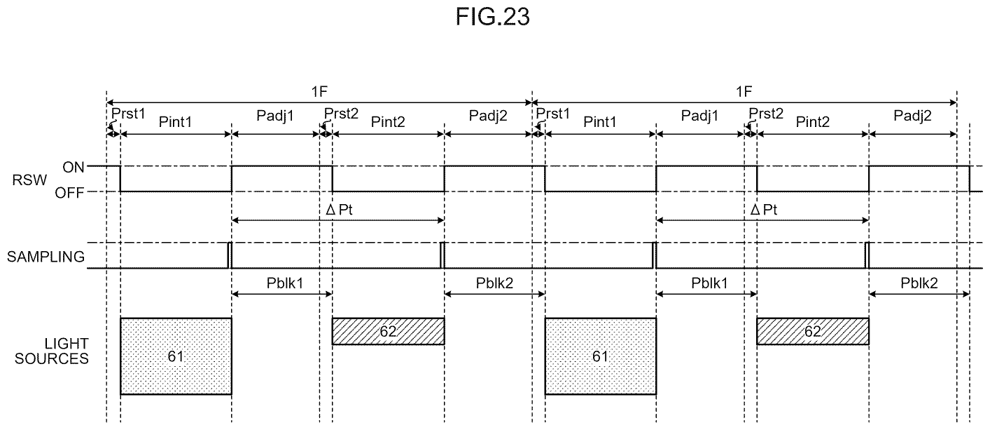

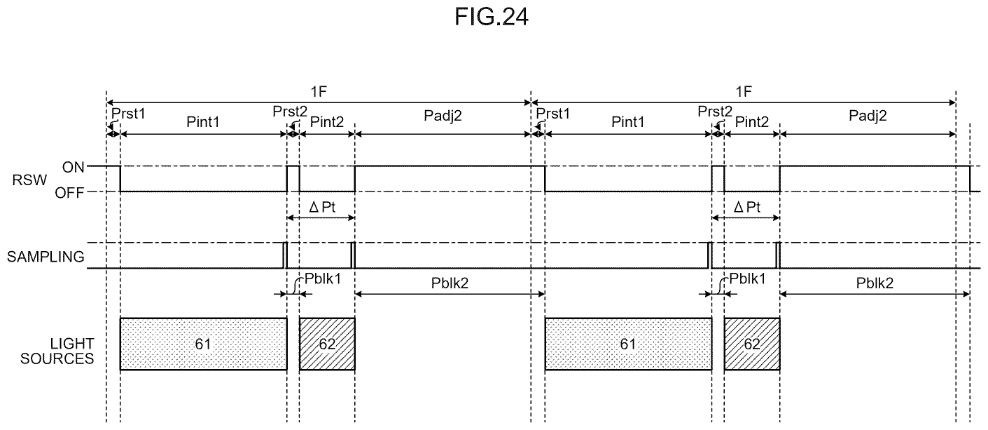

FIG. 23 is an explanatory diagram for explaining an operation example of the detection device according to a comparative example to the third embodiment. FIG. 24 is an explanatory diagram for explaining an operation example of the detection device according to the third embodiment. The length of one frame (1F) period is equalized between the comparative example to the third embodiment illustrated in FIG. 23 and the third embodiment illustrated in FIG. 24 by providing the frame-period adjustment period.

In the examples illustrated in FIGS. 23 and 24, the reset switch RSW is on during the first reset period Prst1, and electric charges of the optical sensor PD and the capacitive element Cfb are reset; and the reset switch RSW is off during the subsequent light emission period of the first light sources 61. As a result, an electric charge corresponding to the emission intensity of the first light sources 61 is stored in the optical sensor PD and the capacitive element Cfb, and the first detection value is acquired at a sampling time before the light emission period of the second light sources 62. Hereinafter, a period after the first reset period Prst1 before the sampling time for acquiring the first detection value is also referred to as a “first integration period Pint1”.

During the subsequent second reset period Prst2, the reset switch RSW is on, and the electric charges of the optical sensor PD and the capacitive element Cfb are reset. During the subsequent light emission period of the second light sources 62, the reset switch RSW is off. As a result, an electric charge corresponding to the emission intensity of the second light sources 62 is stored in the optical sensor PD and the capacitive element Cfb, and the second detection value is acquired at a sampling time before the light emission period of the first light sources 61. Hereinafter, a period after the second reset period Prst2 before the sampling time for acquiring the second detection value is also referred to as a “second integration period Pint2”.

Thus, in the examples illustrated in FIGS. 23 and 24, the first detection value and the second detection value used to calculate the blood oxygen saturation level (SpO₂) are detected for each frame (1F) unit, which is a set of the first reset period Prst1 of the first light detection period, the first integration period Pint1 of the first light detection period, the second reset period Prst2 of the second light detection period, and the second integration period Pint2 of the second light detection period. The first light detection period is a period during which the detection operation using the first light is performed, and the second light detection period is a period during which the detection operation using the second light is performed. In the examples illustrated in FIGS. 23 and 24, the light emission period of the first light sources 61 almost coincides with the first integration period Pint1 in the first light detection period. In the comparative example to the third embodiment illustrated in FIG. 23, the light emission period of the second light sources 62 almost coincides with the second integration period Pint2 in the second light detection period.

In the operation example of the detection device 1 according to the third embodiment illustrated in FIG. 24, the first integration period Pint1 in the first light detection period is longer and the second integration period Pint2 in the second light detection period is relatively shorter than in the comparative example to the third embodiment illustrated in FIG. 23. In the operation example of the detection device 1 according to the third embodiment illustrated in FIG. 24, the emission intensity of the first light sources 61 is made lower and the emission intensity of the second light sources 62 is relatively made higher than in the comparative example to the third embodiment illustrated in FIG. 23.

As a result, the time interval between the first integration period Pint1 of the first light detection period and the second integration period Pint2 of the second light detection period can be made shorter than in the comparative example illustrated in FIG. 23. Therefore, the difference ΔPt in detection timing between the first and the second detection values used to calculate the blood oxygen saturation level (SpO₂) can be made smaller than in the comparative example illustrated in FIG. 23. The first detection value is the detection value acquired in the first integration period Pint1 of the first light detection period, and the second detection value is the detection value acquired in the second integration period Pint2 of the second light detection period. The blood oxygen saturation level (SpO₂) is calculated using the first detection value acquired during the first integration period Pint1 of the first light detection period and the second detection value acquired during the second integration period Pint2 of the second light detection period. By reducing the emission intensity of the first light sources 61 and relatively increasing the emission intensity of the second light sources 62, the luminous efficiencies of the first light sources 61 and the second light sources 62 can be optimized. This optimization enables power saving in the detection operation in the detection device 1 according to the third embodiment.

In the operation example according to the comparative example to the third embodiment illustrated in FIG. 23, after the detection operation using the first light is performed, the first frame-period adjustment period Padj1 is provided between the first integration period Pint1 and the second reset period Prst2; and after the detection operation using the second light is performed, the second frame-period adjustment period Padj2 is provided between the second integration period Pint2 and the first reset period Prst1. The first frame-period adjustment period Padj1 is a period in the first light detection period that does not substantially contribute to the operation to detect the first light, unlike the first reset period Prst1 and the first integration period Pint1. The second frame-period adjustment period Padj2 is a period in the second light detection period that does not substantially contribute to the operation to detect the second light, unlike the second reset period Prst2 and the second integration period Pint2. In the present embodiment, the period between the light emission period of the first light sources 61 and the light emission period of the second light sources 62 is defined as the first blanking period Pblk1. In the present embodiment, the period between the light emission period of the second light sources 62 and the light emission period of the first light sources 61 is defined as the second blanking period Pblk2.

The comparative example to the third embodiment illustrated in FIG. 23 illustrates an example where the first blanking period Pblk1 is almost equal to the second blanking period Pblk2.

In contrast, in the operation example of the third embodiment illustrated in FIG. 24, the first frame-period adjustment period Padj1 is not provided after the detection operation using the first light is performed, and the second frame-period adjustment period Padj2 is provided after the detection operation using the second light is performed. Therefore, the first blanking period Pblk1 does not include the first frame-period adjustment period Padj1, which can shorten the first blanking period Pblk1 relative to the second blanking period Pblk2. Therefore, the difference ΔPt in detection timing between the first and the second detection values used to calculate the blood oxygen saturation level (SpO₂) can be made smaller than in the comparative example illustrated in FIG. 23. By reducing the emission intensity of the first light sources 61 and relatively increasing the emission intensity of the second light sources 62, the luminous efficiencies of the first light sources 61 and the second light sources 62 can be optimized. This optimization enables power saving in the detection operation in the detection device 1 according to the third embodiment.

Also in the operation example according to the third embodiment, the first frame-period adjustment period Padj1 can be provided after the detection operation using the first light is performed. In this case, the first frame-period adjustment period Padj1 is made shorter than the second frame-period adjustment period Padj2 (for example, 1/80 the second frame-period adjustment period Padj2 or shorter, preferably 1/100 the second frame-period adjustment period Padj2 or shorter). This configuration can make the difference ΔPt in detection timing between the first and the second detection values used to calculate the blood oxygen saturation level (SpO₂) smaller than in the comparative example illustrated in FIG. 20.

In the present embodiment, the configuration has been exemplified in which the light emission period of the first light sources 61 almost coincides with the first integration period Pint1 in the first light detection period. However, the light emission period of the first light sources 61 only needs to overlap at least a part of the first integration period Pint1. For example, the first light sources 61 may emit light during a predetermined period of time within the first integration period Pint1, or the first light sources 61 may emit light during a predetermined period of time including the first integration period Pint1. The configuration has also been exemplified in which the light emission period of the second light sources 62 almost coincides with the second integration period Pint2 in the second light detection period. However, the light emission period of the second light sources 62 only needs to overlap at least a part of the second integration period Pint2. For example, the second light sources 62 may emit light during a predetermined period of time within the second integration period Pint2, or the second light sources 62 may emit light during a predetermined period of time including the second integration period Pint2.

While the preferred embodiments of the present disclosure has been described above, the present disclosure is not limited to the embodiments described above. The content disclosed in the embodiments is merely an example, and can be variously modified within the scope not departing from the gist of the present disclosure. Any modifications appropriately made within the scope not departing from the gist of the present disclosure also naturally belong to the technical scope of the present disclosure. At least one of various omissions, substitutions, and changes of the components can be made without departing from the gist of the embodiments and the modifications described above.

What is claimed is:

1. A detection device comprising:
an optical sensor;
a first light source configured to emit first light to the optical sensor;
a second light source configured to emit second light different from the first light to the optical sensor; and
a detection circuit configured to acquire a first detection value when the first light is emitted to the optical sensor and a second detection value when the second light is emitted to the optical sensor, wherein
a light emission period of the second light source is relatively shorter than a light emission period of the first light source.

2. The detection device according to claim 1, wherein the first light is red light, and the second light is infrared light or green light.

3. The detection device according to claim 2, wherein the optical sensor is an organic photodiode.

4. The detection device according to claim 3, wherein the detection circuit has a first light detection period to acquire the first detection value and a second light detection period to acquire the second detection value.

5. The detection device according to claim 4, wherein the detection circuit is configured to acquire the second detection value in the second light detection period after acquiring the first detection value in the first light detection period.

6. The detection device according to claim 5, comprising a plurality of the optical sensors, wherein
the first light detection period comprises:
a first exposure period to emit the first light to the optical sensors; and
a first readout period to read out the first detection value,
the second light detection period comprises:
a second exposure period to emit the second light to the optical sensors; and
a second readout period to read out the second detection value, and
the first detection value and the second detection value are acquired for each frame unit, which is a set of the first exposure period, the first readout period, the second exposure period, and the second readout period.

7. The detection device according to claim 6, wherein the first exposure period is relatively shorter than the second exposure period.

8. The detection device according to claim 7, wherein a second frame-period adjustment period is provided at least between the second readout period and the first exposure period.

9. The detection device according to claim 8, wherein
a first frame-period adjustment period is provided between the first readout period and the second exposure period, and
the first frame-period adjustment period is relatively shorter than the second frame-period adjustment period.

10. The detection device according to claim 9, wherein the first frame-period adjustment period is 1/80 the second frame-period adjustment period or shorter.

11. The detection device according to claim 6, wherein
a plurality of the optical sensors are arranged in a matrix having a row-column configuration in a detection area, and
the detection device comprises:
a drive circuit configured to select the optical sensors arranged in a row direction; and
a selection circuit configured to select the optical sensors arranged in a column direction.

12. The detection device according to claim 6, comprising a plurality of the optical sensors, wherein
the detection circuit comprises a switch circuit configured to select one of the optical sensors.

13. The detection device according to claim 4, wherein
the first light detection period comprises:
a first reset period to reset an electrical charge stored in the optical sensors; and
a first integration period until an electrical charge corresponding to an emission intensity of the first light source is stored in the optical sensor and the first detection value is acquired,
the second light detection period comprises:
a second reset period to reset the electrical charge stored in the optical sensors; and
a second integration period until an electrical charge corresponding to an emission intensity of the second light source is stored in the optical sensor and the second detection value is acquired, and
the first detection value and the second detection value are acquired for each frame unit, which is a set of the first reset period, the first integration period, the second reset period, and the second integration period.

14. The detection device according to claim 13, wherein the first integration period is relatively shorter than the second integration period.

15. The detection device according to claim 14, wherein a second frame-period adjustment period is provided at least between the second integration period and the first reset period.

16. The detection device according to claim 15, wherein
a first frame-period adjustment period is provided between the first integration period and the second reset period, and
the first frame-period adjustment period is relatively shorter than the second frame-period adjustment period.

17. The detection device according to claim 16, wherein the first frame-period adjustment period is 1/80 the second frame-period adjustment period or shorter.

18. The detection device according to claim 13, wherein
the light emission period of the first light source overlaps at least a part of the first integration period, and
the light emission period of the second light source overlaps at least a part of the second integration period.

19. The detection device according to claim 18, wherein
the light emission period of the first light source is provided in a predetermined period in the first integration period, and
the light emission period of the second light source is provided in a predetermined period in the second integration period.

20. The detection device according to claim 18, wherein
the light emission period of the first light source is provided in a predetermined period that includes the first integration period, and
the light emission period of the second light source is provided in a predetermined period that includes the second integration period.
